(19) United States
(12) Patent Application Publication
(10) Pub. No.: US 2006/0123505 A1
(43) Pub. Date: Jun. 8, 2006

(54) Full-length plant cDNA and uses thereof

(75) Inventors: Shoshi Kikuchi, Ibaraki (JP); Naoki Kishimoto, Ibaraki (JP); Kouji Satoh, Ibaraki (JP); Toshifumi Nagata, Ibaraki (JP); Nobuyuki Kawagashira, Tsukuba-shi (JP); Junshi Yazaki, Ibaraki (JP); Masahiro Ishikawa, Ibaraki (JP); Koji Doi, Ibaraki (JP); Jun Kawai, Kanagawa (JP); Yoshihide Hayashizaki, Ibaraki (JP); Yasuhiro Otomo, Ibaraki (JP); Kenichi Matsubara, Osaka (JP); Kazuo Murakami, Ibaraki (JP)

Correspondence Address: KLARQUIST SPARKMAN, LLP, 121 SW SALMON STREET, SUITE 1600, PORTLAND, OR, 97204, US

(73) Assignees: National Institute of Agrobiological Sciences; RIKEN; Foundation for Advancement of International Scienc

(21) Appl. No.: 10/449,902

(22) Filed: May 29, 2003

(30) Foreign Application Priority Data
May 30, 2002 (JP) 2002-203269
Dec. 11, 2002 (JP) 2002-383870

(51) Int. Cl. A01H 5/00; G06F 19/00; C07H 21/04; A01H 1/00; C07K 14/415; C07K 16/18

(52) U.S. Cl. 800/278; 800/320.2; 435/468; 530/370; 435/419; 530/388.1; 536/23.6; 702/20

(57) ABSTRACT

Full-length cDNAs of plants and their uses are provided. Source plants are preferably monocot plants, more preferably poaceous plants, and most preferably rice. Vectors carrying said cDNAs and transformants containing said cDNAs or said vectors, transgenic plants containing said transformants, polypeptides encoded by said cDNAs are also provided. The full-length cDNA clones play important roles in the annotation of correct gene coding region, determination of exons and introns, comprehensive expression analysis on the transcription level and proteome analysis. Furthermore, full-length cDNA clones are industrially useful in producing plants having different properties from those of the wild type due to the inhibition of expression and functional suppression in plant bodies.

### FIELD OF THE INVENTION

The present invention relates to a full-length plant CDNA

### and uses thereof.

### REFERENCE TO TABLES AND A SEQUENCE LISTING

Tables 13 through 17 inclusive and a Sequence Listing are providedinelectronicformatonlyoncompactdiscs, aspermitted under 37 CFR 1.52(e) and 1.821(c). The disc entitled “Tables” (Copy 1 and Copy 2) contains the following files:

| File name | Size (KB) | Date recorded onto disc |
|---|---|---|
| Table 13.doc | 3,770 | May 23, 2003 |
| Table 14.doc | 5,148 | May 23, 2003 |
| Table 15.doc | 234 | May 23, 2003 |
| Table 16.doc | 4,388 | May 23, 2003 |
| Table 17.doc | 858 | May 23, 2003 |

The disc entitled “Sequence List” (Copy 1 and Copy 2) contains the following files:

| File name | Size (KB) | Date recorded onto disc |
|---|---|---|
| 001.txt | 4,688 | Apr. 18, 2003 |
| 002.txt | 4,662 | Nov. 25, 2002 |
| 003.txt | 4,853 | Nov. 25, 2002 |
| 004.txt | 4,832 | Nov. 25, 2002 |
| 005.txt | 4,985 | Nov. 25, 2002 |
| 006.txt | 4,962 | Nov. 25, 2002 |
| 007.txt | 5,030 | Nov. 25, 2002 |
| 008.txt | 5,000 | Nov. 25, 2002 |
| 009.txt | 4,944 | Nov. 25, 2002 |
| 010.txt | 4,981 | Nov. 25, 2002 |
| 011.txt | 5,024 | Nov. 25, 2002 |
| 012.txt | 5,036 | Nov. 25, 2002 |
| 013.txt | 5,662 | Nov. 26, 2002 |
| 014.txt | 7,950 | Nov. 28, 2002 |
| 015.txt | 7,906 | Nov. 28, 2002 |
| 016.txt | 8,018 | Nov. 28, 2002 |
| 017.txt | 8,514 | Nov. 28, 2002 |
| 018.txt | 8,429 | Nov. 28, 2002 |
| 019.txt | 8,350 | Nov. 28, 2002 |
| 020.txt | 8,294 | Nov. 28, 2002 |
| 021.txt | 8,463 | Nov. 28, 2002 |
| 022.txt | 8,449 | Nov. 28, 2002 |
| 023.txt | 499 | Nov. 28, 2002 |

The material on these discs is hereby incorporated by reference in its entirety.

### BACKGROUND OF THE INVENTION

Recently, draft sequences of the rice (indica subspecies) genome (Yu, J. et al., “A Draft sequence of the rice genome (Oryza sativa L. ssp. indica)” Science, 2002, 296, 79-92) and japonica subspecies (Goff, S. A. et al., “A Draft sequence of the ricegenome (Oryza sativa L. ssp. Japonica)” Science, 2002, 296, 92-100), have been published. Both of these draft genome sequences were obtained based on the whole-genome shotgun sequencing method, and therefore they have gaps, and no chromosomal information. In comparison, the International Rice Genome Sequencing Project announced its intention to use the sequencing by the method using BAC/PAC clones to complete most of the rice genome sequence by the end of 2002. The results of partial analysis of rice chromosomes 1 and 4 were published (Sasaki, T. et al., “The genome sequence and structure of rice chromosome 1.” Nature, 2002, 420, 312-316; Q. Feng et al., “Sequence and analysis of rice chromosome 4.” Nature, 2002, 420, 316-320) . The genome sequence data from both rice and Arabidopsis thaliana can be used to compare how monocot and dicot plants differ from each other and how plant genomes differ from animal genomes.

When all of these sequence data are completely obtained, decoding of the rice genome sequence will be accomplished. These sequence data are, however, not sufficient as data for gene function analysis, because the current technology cannot completely predictgene coding regions and other regions based on only the genome sequence data. The full-length cDNA project has been initiated aiming at making up for such data gaps as well as accumulating comprehensive information on the expression of the rice gene and its protein. Information on the full-length cDNA clones greatly contributes to determine annotation, exon and intron of the correct gene-coding region. In addition, such information on these cDNA clones is important for the exhaustive expression analysis at the transcriptional level and proteome analysis. The results of the full-length cDNA project on Arabidopsis have already been published (Seki, M. et al., “Functional Annotation of a Full-Length Arabidopsis cDNA Collection.” Science, 2002, 296, 141-145).

### SUMMARY OF THE INVENTION

Even today when decoding of the rice genomic DNA is coming to an end, we are still in great need of isolation and characterization of the full-length cDNA of rice.

These full-length cDNA clones can be used for various purposes. They play important roles in the annotation of correct gene coding regions, exon-intron determination, comprehensive expression analysis at the transcription level and analysis of proteome. Furthermore, full-length cDNAs are industrially useful in creating plant bodies showing a phenotype that is different from the wild type as a result of inhibiting their expression and function in the plant bodies.

Diverse uses of these full-length cDNA clones can be mentioned. First, alignment of these clones togenome sequences enables to check the fidelity of computer prediction of gene coding regions from the genome sequence, such as, a transcription initiation point, exons, introns, and a transcription termination point. In contrast, this fidelity checking leads to the improvement of gene coding region prediction program. Furthermore, information of full-length cDNA clones is combined with results of comprehensive analysis of expression profiles at the transcriptional level using microarrays such, to predict promoter and transcription regulatory regions of genes of interest in the genome sequence.

Furthermore, comparison between full-length cDNA clone sequence information and sequences adjacent the insertion site of insertion-mutants such as transposon insertion mutants, enables to find a plant whose gene corresponding to said clone is disrupted. The function of said gene can be predicted from the phenotype of the plant. It is also possible to express the protein encoded by the full-length cDNA clone using various systems such as Escherichia coli, yeast, in vitro system so as to investigate the biochemical function and conformation of the protein, thereby elucidating overall its functions. Furthermore, proteins that interact with said protein can be found by using the yeast two-hybrid system and such to clarify a part of a biological network in vivo.

Given this, an objective of the present invention is to provide the full-length cDNA clones of plants. Another objective of this invention is to modify plants using the cDNAs thus isolated and characterized.

The present inventors collected 3′-EST sequences of 175,642 full-length cDNA clones using two different methods, namely, the oligo-capping and biotinylated CAP trapper methods. These clones were clustered into 28,469 nonredundant groups, and all representative clones from each group were completely sequenced with 99.98% fidelity. As a result of homology searches, 21,596 (75.86%) of these representative full-length cDNA clones (28,469) were annotated. ORFs were present in 28,332 clones; among them 24,507 clones had ORFs containing 100 or more amino-acid residues. As a result of attempting to map the28,469 full-length cDNA clones, 18,933 transcription units (TU) were mapped to the indica draft genome. Of the said full-length cDNA clones, 18,900 clones (12,996TU) had a homology to genes (27,288) of Arabidopsis predicted from its genome sequence. Thus, the present inventors succeeded in comprehensive collection, grouping, sequencing and functional annotation of full-length cDNA clones from rice.

That is, the present invention relates to full-length cDNAs of plants and uses thereof, more specifically to:

[1] An isolatedplant-derivednucleic acid, wherein saidnucleic acid is selected from the group consisting of:

(a) a nucleic acid encoding a protein comprising an amino acid sequence set forth in any one of SEQ ID NOs: 28470 through 56791;

(b) a nucleic acid containing the coding region of a nucleotide sequence set forth in any one of SEQ ID NOs: 1 through 28469;

(c) a nucleic acid encoding a protein comprising an amino acid sequence set forth in any one of SEQ ID NOs: 28470 through 56791 wherein one or more amino acids are substituted, deleted, inserted and/or added; and

(d) a nucleic acid hybridizing to a nucleic acid comprising a nucleotide sequence set forth in any one of SEQ ID NOs: 1 through 28469 under stringent conditions.

[2] The nucleic acid according to [1], wherein said nucleic acid is derived from rice.

[3] An isolated DNA molecule selected from the group consisting of:

(a) a DNA molecule encoding an antisense RNA complementary to a transcript of the DNA molecule of [1] or [2];

(b) a DNA molecule encoding RNA having ribozyme activity to specifically cleave a transcript of the DNA of [1] or (2];

(c) a DNA molecule encoding RNA inhibiting the expression of the DNA of [1] or [2] via an RNAi effect at the time of expression of said DNA in plant cells; and

(d) a DNA molecule encoding RNA inhibiting the expression of the DNA of [1] or [2] by the co-suppression effect at the time of expression of said DNA in plant cells.

[4] A vector containing the nucleic acid of anyone of [1] through [3] .

[5] A transformed plant cell maintaining the nucleic acid of any one of [1] through [3] or the vector of [4].

[6] A transformed plant body containing the transformed plant cell of [5].

[7] A progeny or clone of the transformed plant body of [6].

[8] A propagation material of the transformed plant body of [6] or [7].

[9] A method of producing the transformed plant body of [6], wherein said method comprises the step of transducing the DNA of any one of [1] through [3] or the vector of [4] into plant cells to regenerate a plant body from said plant cells.

[10] A protein encoded by any one of the nucleic acids of [1].

[11] A method of producing the protein of [10] comprising the following steps:

(1) transducing any one of the nucleic acids of [1] or a vector containing said nucleic acid into cells capable of expressing said nucleic acid so as to obtain a transformant;

(2) culturing said transformant; and

(3) recovering the protein of [10] from the culture of the step (2).

[12] An antibody binding to the protein of [10].

[13] A rice gene database comprising sequence information selected from the group consisting of:

(a) one or more amino acid sequences selected from SEQ ID NOs: 28470 through 56791;

(b) one or more nucleotide sequences selected from SEQ ID NOs: 1 through 28469; and

(c) both (a) and (b).

[14] A method of determining the transcriptional regulatory region comprising the steps of:

(1) mapping the nucleotide sequence of any one of SEQ ID NOs: 1 through 28,469 to the rice genome nucleotide sequence, and

(2) determining the transcriptional regulatory region of the gene mapped in the step (1) which contains the transcriptional regulatory region found on the 5′-side of the 5′ most end of the mapped region.

### BRIEF DESCRIPTION OF THE DRAWING

FIG. 1 represents the results of detailed comparison of CDS1 region at the exon and intron levels. One complete coding sequence (CDS) has been predicted in the BACO-10 kb region; this figure shows introns and exons in the region and relationship between two cDNA clones of the present invention that have been mapped to the BACO-10 kb region.

### DETAILED DESCRIPTION OF THE INVENTION

The present invention provides full-length cDNA clones derivedfromplants. Nucleotide sequences of full-length cDNA clones isolated from a rice plant by the present inventors are set forth in SEQ ID NOs: 1 through 28,469 and amino acid sequences of proteins encoded by these cDNAs are shown in SEQ ID NOs: 28,470through56,791. Correspondences of the nomenclature of each clone, SEQ ID NOs of nucleotide sequences, initiation and terminationpoints of ORFs, and SEQ ID NOs of amino acid sequences encoded in ORFs are set forth in the “List of Clones” at the end of this specification.

As used herein, an “isolated nucleic acid” is a nucleic acid the structure of which is not identical to that of any naturally occurring nucleic acid or to that of any fragment of a naturally occurring genomic nucleic acid spanning more than three genes. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment;_and (d) a recombinant nucleotide sequence that is part of a hybrid gene, i.e., a gene encoding a fusion protein. Specifically excluded_from this definition are nucleic acids present in random, uncharacterized mixtures of different DNA molecules, transfected cells, or cell clones, e.g., as these occur in a DNA library such as a CDNA or genomic DNA library.

Of 28,469 clones, those with the longest ORF encoding 100 or more amino acid residues were examined for what functional domains they had against the InterPro DB, yielding a total of 3491 InterPro domains. Comparison of the search results of rice cDNA clones obtained in the present invention with those of cDNAs from Arabidopsis thaliana, Caenorhabditis elegans, Drosophila melanogaster, Homo sapiens, Saccharomyces cerevisiae and S. pombe yielded the following characteristic InterPro domains.

Found commonly in eukaryotes (Table 4a)

Found specifically or frequently in rice (Table 4b and 4c)
Pollen allergic protein domain
Expressed organ-specifically (e.g., serine protease inhibitor in seeds)
Environmental stress-inducible proteins (anti-freeze
protein, ABS/WDS-inducible anti-drought protein, etc.)

Found specifically and frequently in Arabidopsis thaliana

(Table 4d)
TIR domain

Transcription factors play important roles in the gene expression control of living organisms. Controlling the transcription factor activity is none other than controlling the gene expression. Therefore, genes encoding transcription factors are useful in the control of the gene expression in plants.

The InterPro search for full-length cDNAs of the present invention yielded 1336 transcription factor clones classified into 18 DNA binding domains, and the details are shown in Table 5. Clones predicted as the transcription factor are shown in the “List of clones predicted as the transcription factor” categorized into the respective classes at the end of this specification. As a result of this classification, Zn finger-type transcription factors are dominant, followed by Myb-type factors; these constitutions are shared with Arabidopsis. Of the clones predicted as a transcription factor, those classified into Zn finger-type were collected and subclassified in the “List of clones predicted as Zn finger type” at the end of this specification.

Control of a transcription factor expression enables to control the expression of a gene transcriptionally regulated by said transcription factor. For example, a plant body produced by transducing an antisense sequence of a transcription factor involved in the transcription of a gene causing an undesirable phenotype enables to suppress the action of said transcription factor. It is also possible to competitively inhibit the action of a transcription factor by transducing into cells a nucleic acid imitating the recognition sequence for the transcription factor as a decoy nucleic acid. The recognition sequence for transcription factors encoded by full-length cDNA of the present invention can be determined by the footprinting method or gel shift assay. On the other hand, the activity of a transcription factor involved in expression of a desirable phenotype can be enhanced by transducing a gene encoding the transcription factor.

Plant membrane proteins play important roles in the interaction between cells, absorption of nutrients from the extracellular environment, recognition and infection by viruses, etc. For example, the transporter present in the plasma membrane is closely associated with the salt tolerance of plants. Therefore, full-length cDNA clones encoding the plant membrane protein is useful in controlling various properties of plants.

The MEMSAT program was used to predict transmembrane domains of proteins encoded by full-length cDNA clones of the present invention. As shown in Table 6, 6,280 clones, which account for 22.1% of the whole full-length cDNA clones, had two or more transmembrane-spanning domains. When the direction of the transmembrane domain whose N-terminus is intracellular is referred to as IN, and whose N-terminus is extracellular OUT, the number of transmembrane spanning domains in IN direction and that in OUT direction was about the same, although, in some instances, the number of either one direction is predominant over the other, depending on the number of transmembrane spanning.

Furthermore, intracellular localization of proteins, encoded by full-length cDNA clones of the present invention, was analyzed using the pSORT program. Among those proteins with ORF encodding 100 or more amino acid residues, those encoded by 18,166 clones were predicted tobe localized intracellularly. Table 7 shows the putative target organelles, the number of clones encoding proteins that are predicted to belocalized, and the ratio (%) of said number of clones to 18,166 clones. The major organelle where the proteins are localized is nucleus, accounting for 20.0% of clones. Second major organelles include plasma membrane, cytoplasm, endoplasmic reticulum (ER), and microbody, and each accounted for about 10% of the clones analyzed.

Homology search was performed between known genes and nucleotide sequences of the full-length cDNA clones or their amino acid sequences of the present invention as query sequences. BLASTN search revealed that 2603 cDNA clones were identical to already-known rice genes. These clones were classified into the identical rice genes. BLAST X search revealed that 5607 clones were homologous to rice homolog genes, 12 527 clones to genes of other plants than rice, and 859 clones to genes of organisms other than plants. These results were used to functionally classify 21,596 (75.86%) of 28,469 full-length cDNA clones of the present invention. The identical rice genes or the already known genes found to be homologous to genes of rice and plants other than rice include the following genes.

<Rice Ribosomal RNA Gene>

Fumio Takaiwa, Shoshi Kikuchi and Kiyoharu Oono, “Cloning of rice ribosome RNA (rRNA) gene (III). Full-length nucleotide sequence of rice rRNA gene (abstract, oral presentation),” Ikushugaku Zasshi (Breeding Science), 1985 April, Vol. 35 (suppl. 1), pp. 214-215.
   <Rice Storage Protein Gene, Glutelin>
Fumio Takaiwa, Shoshi Kikuchi and Kiyoharu Oono, “Cloning and structural analysis of rice storage protein glutelin cDNA (Abstract, oral presentation) ,” Abstracts of the annual meeting, The Molecular Biology Society of Japan, 1985 December, Vol. 8, p. 54.
Fumio Takaiwa, Shoshi Kikuchi and Kiyoharu Oono, “Heterogeneity of rice storage protein glutelin mRNA (Abstract, oral presentation),” Abstr. Annu. Meet. Mol. Biol. Soc. Jap., 1986 December, Vol. 9, p. 222.
Fumio Takaiwa, Hiroyasu Ebinuma, Shoshi Kikuch and Kiyoharu Oono, “Structure of rice seed storage protein glutelin nuclear gene (Abstract, oral presentation),” Abstr. Annu. Meet. Mol. Biol. Soc. Jap., 1987 December, Vol. 10, p. 139.
Fumio Takaiwa, Akira Kato, Shoshi Kikuchi and Kiyoharu Oono, “Expression of rice storage protein glutelin gene group—identificationof tissue specific expression region (Abstract, oral presentation),” Ikushugaku Zasshi (Breeding Science), 1988 October, Vol. 38 (Suppl. 2), pp. 154-155.
Fumio Takaiwa, Akira Kato, Shoshi Kikuchi and Kiyoharu Oono, “Structure and expression control of rice storage protein glutelingene group (Oral presentation, abstract),” Abstracts of 11th Annual meeting, The Molecular Biology Society of Japan, 1988 December, p. 284.
Fumio Takaiwa, Shoshi Kikuchi and Kiyoharu Oono, “Structure and expression of rice storage protein glutelin genes (Oral presentation, abstract),” Abstr. 2nd Int. Congr. Plant Mol. Biol., 1988 November, p. 325.
Shoshi Kikuchi, Fumio Takaiwa and Kiyoharu Oono, “Gene expression analysis in rice cultured cells—analysis of protein synthesis in rice callus using two-dimensional electrophoresis (Oral presentation, abstract),” Ikushugaku Zasshi (Breeding Science), 1985 April, Vol. 35 (Suppl. 1), pp. 14-15.
   <Rice pRB301 and pRB401 DNAs>
Shoshi Kikuchi, Yoshio Kaneko, Takao Komatsuda, Fumio Takaiwa and Kiyoharu Oono, “DNA analysis in rice cultured cells—isolation and characterization of DNA fragments whose copy numbers are significantly different between rice embryo and callus (oral presentation, abstract),” Ikushugaku Zasshi (Breeding Science), 1985 September, Vol. 35 (suppl. 2), pp. 102-103.
Shoshi Kikuchi, Fumio Takaiwa and Kiyoharu Oono, “Analysis of rice genes—analysis of DNA fragments whose copy numbers are varied in cultured cells and differentiated cells (oral presentation, abstracts), ” Abstr. Annu. Meet. Mol. Biol. Soc. Jap., 1985 December, Vol. 8, p. 56.
Shoshi Kikuchi, Fumio Takaiwa and Kiyoharu Oono, “Analysis of rice genes—analysis of DNA whose copy numbers are reversibly fluctuated in differentiation and dedifferentiation states (Oral presentation Abstr.),” Abstr. Annu. Meet. Mol. Biol. Soc. Jap., 1985 December, Vol. 9, p. 224.
Kiyoharu Oono, Shoshi Kikuchi and Fumio Takaiwa, “DNA amplification and diminution in rice callus culture,” Abstr. 6th Intnl. Cong. Plant Tissue Culture Society, 1986 August, p. 287.
Shoshi Kikuchi, Fumio Takaiwa and Kiyoharu Oono, “Analysis of nucleotide sequence of variable copy number DNAin rice (oral presentation, abstr.),” Ikushugaku Zasshi (Breeding Science), 1987 Octover, Vol. 37 (Suppl. 2), pp. 122-123.
Shoshi Kikuchi, Fumio Takaiwa and Kiyoharu Oono, “Analysis of nucleotide sequence of variable copy number DNA in rice (oral presentation, abstr.)” Abstr. Annu. Meet. Mol. Biol. Soc. Jap., 1987 November, Vol. 10, p. 140.
Shoshi Kikuchi, Taiichi Ogawa, Fumio Takaiwa and Kiyoharu Oono, “Analysis of redundant DNA sequence transcribed in rice (Oral presentation Abstr.), ” Ikushugaku Zasshi (Breeding Science), 1988 Octover, Vol. 38 (suppl. 2), pp. 118-119.
   <Rice Drought-Responsive Genes>
Shoshi Kikuchi, M. Soliman, Shin Dong-Hyun, Kazunari Maruta, Fumio Takaiwa and Kiyoharu Oono, “Gene expression analysis in rice dry callus—Cloning and characterization of the genes which specifically express in rice callus culture under dried condition (oral presentation, abstr.),” Abstr. Annu. Meet. Mol. Biol. Soc. Jap. 13th, 1990 December, p. 262.
Shoshi Kikuchi, M. Soliman, Shin Dong-Hyun, Kazunari Maruta, Fumio Takaiwa and Kiyoharu Oono, “Analysis of genes which specifically express in rice dry callus culture (oral presentation abstr.),” Ikushugaku Zasshi (Breeding Science), 1990 November, Vol. 40 (suppl. 2), p. 20-21.
Shoshi Kikuchi, M. Soliman, Shin Dong-Hyun and Kiyoharu Oono, “Cloning and characterization of the genes which express under dried condition of rice callus (Accumulation of MRNA discovered in rice callus under dried conditions) (oral presentation abstr.), ” 20th Annu. Meet. Mol. Cell Biol. at Intnl. Congr. Mol. Biol. (Keystone Symposium), 1991 January, p. 58.
Shoshi Kikuchi, Kazunari Maruta and Kiyoharu Oono, “Analysis of gene specifically expressed in rice dry callus II—Giant transcript discovered in mature seeds and dried rice callus culture (oral presentation abstr.),” Ikushugaku Zasshi (Breeding Science), 1991 April, Vol. 41 (Suppl. 1), pp. 136-137.
Shoshi Kikuchi and Kiyoharu Oono, “Giant transcript of drought-specific gene discovered in mature seeds and dried callus culture of rice (oral presentation abstr.),” 4th Plant Mol. Biol. Symp., 1991 January, p. 37.
Shoshi Kikuchi and Kiyoharu Oono, “Cloning and characterization of genes specifically expressed in rice callus (Analysis of genes expressed in rice callus) (Oral presentation, abstr.),” Intnl. Workshop Rice Mol. Biol., 1991 August, p. 35.
Shoshi Kikuchi and Kiyoharu Oono, “Cloning and characterization of the genes which specifically express in callus of rice (Oral presentation abstr.) ,” Abst. 3rd Congr. Int. Soc. Plant. Mol. Biol., 1991 Octover, p. 872.
Shoshi Kikuchi, Kazumaru Miyoshi, Kazunari Maruta and Kiyoharu Oono, “Rice callus cDNA clones for the characterization and understanding of calli in molecular level (oral presentation abstr.), German-Japanese Work-shop Plant Culture, Breeding and Formation of Phytochemicals, Abstr, 1992 February, p. 35.
Shoshi Kikuchi, Kazumaru Miyoshi, Kazunari Maruta and Kiyoharu Oono, “cDNA cloning from rice callus I. Classification of clones based on the expression specificity and analysis of nucleotide sequences (Oral presentation, abstract),” Ikushugaku Zasshi (Breeding Science), 1992 April, Vol. 42 (suppl. 1), pp. 206-207.
Kazumaru Miyoshi, Shoshi Kikuchi, Kazunari Maruta, Tadao Naito and Kiyoharu Oono, “Expression analysis of rice callus cDNA in long-term subcultured cells (oral presentation abstr.),” Ikushugaku Zasshi (Breeding Science), 1992 April, Vol. 42 (suppl. 1), pp. 208-209.
   <Rice Heat-Shock Inducible Genes>
Kazumaru Miyoshi, Shoshi Kikuchi, Kazunari Maruta, Kiyoharu Oono and Tadao Naito, “Analysis of heat shock protein-like protein gene expressed in rice callus (oral presentation abstr.),” Ikushugaku Zasshi (Breeding Science), 1992Octover, Vol. 42 (suppl. 2), pp. 196-197.
   <Rice SNF-1-Like Protein Gene, Osk1>
Kanegae, H., H. Funatsuki, S. Kikuchi and M. Takano, “Differential expression of the rice snf-1 related protein kinase gene family.” Abs. Int. Congr. Plant Mol. Biol. 5th, p. 147.
Takano, M., H. Kanegae, and S. Kikuchi, “Genome structure of SNF-related protein kinase genes in rice.” Abs. Annu. Meet. Mol. Biol. Soc. Jpn. 20th, 1997, 4-EH-P-065.
Takano, M., H. Kanegae, K. Miyoshi, M. Mori, S. Kikuchi and Y. Nagato, “Rice has two distinct classes of protein kinase genes related to SNF1 of Saccharomyces cerevisiae, which are differently regulated in the early seed development.”Interact, Intersect. Plant Signal Pathw, 1999, 60.
Kanegae, H, S. Kikuchi and M. Takano, “Analysis of promoter activity of OSK genes in rice.” Plant Cell Physiol., 1998, 39 (suppl), p. 125.
Takano, M., H. Kanegae, K. Miyoshi, M. Mori, S. Kikuchi and Y. Nagato, “Rice has two distinct classes of protein kinase genes related to SNF1 of Saccharomyces cerevisiae, which are differently regulated in the early seed development.” Plant Cell Physiol., 1999, 40 (suppl), p. 79.
   <Rice Blue Light Receptor NPH1-Like Protein Gene>
Kanegae, H., M. Tahira, S. Kikuchi, K. Yamamoto, M. Yano, T. Sasaki, K. Kanegae, M. Wada and M. Takano, “Identification of NPH1 homologs in rice.” Abs. Annu. Meet. Mol. Biol. Soc. Jap. 21st, 1999, p. 274.
   <Rice Flower Organ Formation Gene, OSSUPL>
Masaki Mori, Hiroshi Takatsuji, Hiromi Kanegae, Toshifumi Nagata, Yuriko Shibata and Shoshi Kikuchi, “Cloning and structural analysis of rice SUPERMAN gene.” Abs. Annu. Meet. Mol. Biol. Soc. Jpn., 1997, p. 475.
Masaki Mori, Hiroshi Takatsuji, Hiromi Kanegae, Toshifumi Nagata, Yurikio Shibata and Shoshi Kikuchi, “Isolation and expression analysis of genomic DNA of rice SUPERMAN gene (RSUP).” Ikushugaku Zasshi (Breeding Science), 1998, 48 (suppl. 1), p. 28.
Masaki Mori, Hiroshi Takatsuji, Hiromi Kanegae, Toshifumi Nagata, Yuriko Shibata and Shoshi Kikuchi, “Isolation and characterization of a rice gene encoding a zinc-fingerprotein related to Arabidopsis SUPERMAN.” Abstracts of 15th International Congress on Sexual Plant Reproduction, 1998, p. 96.
   <Rice Brassinosteroid Synthase Gene, OsBR6ox>
Masaki Mori, Hisako Ooka, Kazuhiko Sugimoto, Kouji Sato, Hirohiko Hirochika, Koji Yamamoto and Shoshi Kikuchi, “Analysis of dwarf mutant strain whose phenotype is recovered by brassinolide.” Proceeding of the Annual Meeting, The Japanese Society of Plant Physiologists, 2002, p. 225.
Masaki Mori, Takahito Nomura, Hisako Ooka, Masumi Ishizaka, Takao Yokota, Kazuhiko Sugimoto, Ken Okabe, Kouji Sato, Koji Yamamoto, Hirohiko Hirochika and Shoshi Kikuchi, “Rice extremely dwarf mutant brd 1 is a brassinosteroidbiosynthesis mutant.” Ikushugaku Kenkyuu (Breeding Research), 2002, 4 (suppl. 2), p. 352.
Masaki Mori, Hisako Ooka, Takahito Nomura, Masumi Ishizaka, Takao Yokota, Kazuhiko Sugimoto, Kouji Satoh, Hirohiko Hirochika and Shoshi Kikuchi, “Isolation and characterization of a rice dwarf mutant with the defect in the brassinolide biosynthesis” Abstracts of 13th Congress of the Federation of European Societies of Plant Physiology, 2002, p. 233.
   Publications
   <Rice Storage Protein Glutelin Genes>
Fumio Takaiwa, Shoshi Kikuchi and Kiyoharu Oono, “The structure of rice storage protein glutelin precursor deduced from cDNA”. (FEBS. Lett., 1986 September 206(1), pp. 33-35.
Sequencing of cDNA proved that the glutelin precursor consisted of 49 amino acid residues, arranged in order of signal peptide-acidic subunit-basic subunit.
Fumio Takaiwa, Shoshi Kikuchi and Kiyoharu Oono, “Ariceglutelin gene family—A major type of glutelin mRNAs can be divided into two classes”. Mol. Gen. Genetics, 1987 July 208, pp. 15-22.
Analysis of cDNA clones of rice glutelin gene revealed that they could be classified into 2 classes based on the differences in the restriction enzyme map and expression time, etc.
Fumio Takaiwa, Hiroyasu Ebinuma, Shoshi Kikuchi and Kiyoharu Oono, “Nucleotide sequence of a rice glutelin gene” FEBS. Lett., 1987 August 221(1), pp. 43-47.
Genomic DNA of rice glutelin gene was cloned to elucidate its entire structure.
   <Rice pRB301 and pRB401 DNAs>
Shoshi Kikuchi, Fumio Takaiwa and Kiyoharu Oono, “Variable copy number DNA sequences in rice” Mol. Genetics, 1987 December 210, pp. 7373-380.
Rice nuclear DNA whose copy number reversibly changes during cellular differentiation and dedifferentiation, was cloned to elucidate its structure.
   <Rice Gravity Stress Responsive Gene>
Kwon, S. T., Shoshi Kikuchi and Kiyoharu Oono, “Molecular cloning and characterization of gravity specific cDNA in rice (Oryza sativa L.) suspension callus” Jpn. J. Genet., 1992 August 67, pp. 335-348.
Rice gene specifically expressed under 450,000× g high gravity condition was isolated and characterized.
Shoshi Kikuchi, Kazumaru Miyoshi, kazunari Maruta and Kiyoharu Oono, “cDNA clones of rice callus for the analysis of plant tissue culture problems,” Proc. Plant Tissue Culture and Gene Manipulation for Breeding and Formation of Phytochemicals, German-Japanese Work-shop Plant Culture, Breeding and Formation of Phytochemicals, Abstr., National Institute of Agrobiological Sciences), 1992 July, pp. 173-178.
   <Rice SNF-1-Like Protein Gene>
Takano, M., H. Kanegae, H. Funatsuki and S. Kikuchi, “Rice has two distinct classes of protein kinase genes related to SNF1 of Saccharomryces cerevisiae, which are differently regulated in seed development.” Mol. Gen. Genetics, 1998, 260, pp. 388-394.
   <Rice Fe-Deficiency-Responsive Protein Gene>
Takashi Negishi, Hiromi Nakanishi, Junshi Yazaki, Naoki Kishimoto, Fumiko, Fujii, Kanako Shimbo, Kimiko Yamamoto, Katsumi Sakata, Takuji Sasaki, Shoshi Kikuchi, Satoshi Mori and Naoko K. Nishizawa, “cDNA microarray analysis of gene expression during Fe-deficiency stress in barley suggests that polar transport of vesicles is implicated in phytosiderophore secretion in Fe-deficient barley roots,” The Plant Journal, 2002, 30, pp. 83-94.
   <Rice Brassinosteroid Synthase Gene, OsBR6ox>
Masaki Mori, Takahito Nomura, Hisako Ooka, Masumi Ishizaka, Takao Yokota, Kazuhiko Sugimoto, Ken Okabe, Hideyuki Kaj iwara, Kouji Satoh, Koji Yamamoto, Hirohiko Hirochika and Shoshi Kikuchi, “Isolation and characterization of a rice dwarf mutant with a defect in brassinosteroid biosynthesis” Plant Physiology, 2002, Vol.130, pp. 1152-1161.

Additional homology search was performed between amino-acid sequences encoded by ORFs of full-length cDNA clones of the present invention and those encoded by the predicted CDS of the Arabidopsis genome nucleotide sequence. As shown in Table 8, 18,900 full-length cDNA clones (12,996 TU) yielded hits, confirming the homology of the aforementioned ORF amino acid sequences with those Arabidopsis with high probability. These results substantiated a high reliability of amino acid sequences obtained in this invention. Furthermore, it is highly reliable that cDNAs of the present invention have full-length, which makes their ORFs also reliable.

“BLAST search results” at the end of this specification lists combinations of clones which showed the highest homology among the 28,469 clones searched by BLAST N and BLAST X. Rice genes highly homologous to known genes whose functions are known, would function similar to the homologous known genes.

For example, the following clones were found to have the serpin or serine protease domain:

002-145-A10 4867 1: serine protease inhibitor, and

001-125-A10 4868 1: serpin

Proteins having these domains are involved in vermin tolerance (see the website of National Center for Biotechnology information, http://www.ncbi.nlm.nih.gov/entrez/query.fcgi?cmd=Retrieve &db=PubMed&list uids=12354191&dopt=Abstract). Therefore, the above clones are useful in breeding plants having disease and vermin tolerances.

The clone, J023033I19 5505 1, was confirmed to have the heavy metal binding domain. Therefore, this clone can be used as a gene for conferring the heavy metal tolerance on plants, or the heavy metal absorption capability on plant cells.

The following clones were found to have the domain associated with ethylene receptors, which are phytohormone receptors and involved in controlling the maturation and deterioration of plants.

J023034P21 156 1: two-component response regulator and

J023056E19 155 1: two-component sensor molecule.

Proteins having the following domains are assumed to be so-called G-proteins, which are involved in the signal transduction and are useful in the regulation of signal transduction system in plants.

RAB small monomeric GTPase,

RAS small monomeric GTPase, and

GTPase small monomeric GTPase.

Proteins having the following domains are expected to be involved in the signal transduction system.

Inositol-3-phosphate synthase, and calcium ion binding.

Clones (e.g., 002-143-H11) having the above domains are useful in controlling the signal transduction in plants.

Gene ontology (GO) is useful to clarify the function of a gene based on the results of motif and homology searches using protein informatics analysis against the InterPro database. Construction of GO enables to systematically comprehend functions of known genes detected by a homology search.

Motif search results predict functional domains of proteins encoded by cDNAs of the present invention. Homology search results predict functions of known genes having homology to full-length cDNAs of this invention. However, functions of known genes described as DEFINITION in the homology search results below arevariously expressed. To comprehend functions of each gene from expressions of functions, the expressions should be unified as much as possible. GO is a tool for comprehending gene functions by replacing expressions actually assigned to each gene with a unified expression. In other words, the use of GO makes it possible to comprehend homology search results by the unified expression terms. GO terms have been attached to the GenBank report, InterPro domain names, and Arabidopsis genes. Therefore, if GO terms are assigned to the records of the database to which full-length cDNAs of the present invention showed homology, GO terms can also be assigned to full-length cDNA clones of this invention based onhomology search results. Full-length cDNA clones of this invention were classified based on the GO terms.

The unified expression assigned to genes using GO is referred to as GO term. It is classified into a number of categories. In other words, each category may include plural GO terms. Furthermore, categories are divided into subcategories based on different aspects, and one GO term may be included in two or more categories.

GO terms assigned to full-length cDNA clones of the present invention are shown at the end of this specification. Furthermore, the names of categoriess and the number of constitutive GO terms in each category are listed in Tables 10-12.

The total number of GO terms associated with “biological processes” in the full-length cDNA clones of the present invention was 18,485. Tables 9-12 show categorization of these clones. The largest class was “Unclassified,” accounting for about ½ of the total terms, followed by “Metabolism,” “Transport,” and “Translation”. GO terms associated with “function” were assigned to 10,942 full-length cDNA clones, and the total number of these GO terms was 16,853. These GO terms could not be classified exclusively, and the most frequently observed GO “function” term was “enzyme”. GO terms associated with “cellular component” were assigned to 3629 full-length cDNA clones, and the total number of the terms was 3637.

Relationship between GO terms contained in each category and clones to which the GO terms are assigned is shown in the “List of clones included in each Gene Ontology category” at the end of this specification. The function of each clone can be found from GO terms shown in the list. Representative GO terms will be specifically described below. The largest number of clones was classified into the category “Enzyme”. GO function term “Enzyme” includes many industrially useful enzymes as listed below. When protein functions are predicted by Motif search results, prediction as being enzymatic activity is usually highly accurate. In other words, proteins whose functions associated with enzyme are predicted by Motif search results would have that function actually. Therefore, proteins to which GO term included in category “Enzyme” attached to the InterPro domain names (results of functional domain search) would have the activity corresponding to that GO term. Since the GO terms included in the category “Enzyme Inhibitor” also designate functions to control enzyme actions, proteins to which these GO terms are assigned include many of those having useful functions like proteins to which “Enzyme” Go terms are assigned.

1,3-beta-Glucan synthase:

Chitinase:

This enzyme is involved in disease tolerance and vermin tolerance of plants. Therefore, genes encoding proteins having this enzyme activity are useful in breeding disease tolerant or vermin tolerant plants.

1l-Aminocyclopropane-1-carboxylate synthase:

This enzyme is involved in the synthesis of ethylene, a phytohormone. Therefore, a gene encoding a protein having this enzyme activity is useful in breeding environmental stress-tolerant plants.

Alpha, alpha-trehalase:

Alpha, alpha-trehalose-phosphate synthase (UDP-forming):

These enzymes are involved in the synthesis of trehalose, which is an element regulating cryotolelance of plants. Therefore, a gene encoding a protein having either one of these enzyme activities is useful in breeding cryotolerant plants.

Alpha-amylase:

Beta-amylase:

These enzymes are involved in degrading plant storage starch. Therefore, genes encoding these proteins are useful in the artificial regulation of germination.

Aspartate kinase:

Glutamate synthase:

These enzymes are involved in the nitrogen metabolic system of rice. Therefore, genes encoding proteins having these enzyme activities can be used in breed improvement.

Caspase:

This is the enzyme involved in cell death of plant. Therefore, genes encoding proteins having this enzyme activity are useful in the artificial induction of apoptosis in plants.

Catalase:

Copper, zinc superoxide dismutase:

Ferredoxin reductase:

Glutathione peroxidase:

Glutathione synthase:

Peroxidase:

Superoxide dismutase:

These enzymes play important roles in the plant response to oxidation stresses. Therefore, genes encoding proteins having these enzyme activities are important in breeding oxidation stress tolerant plants.

Hexokinase:

This enzyme is involved in the accumulation of storage substances in rice. Therefore, genes encoding proteins having this enzyme activity are useful in the improvement of phenotypes associated with plant storage substances.

O-Methyltransferase:

This enzyme is involved in the syntheses of secondary metabolites in plants. Therefore, genes encoding proteins having this enzymatic activity are useful in producing pharmacological substances.

Phosphoenolpyruvate carboxylase:

This enzyme has an important role in the C4 photosynthesis. Therefore, genes encoding proteins having this enzyme activity may be used in improvement of photosynthesis efficiency.

Phospholipase C:

This enzyme is involved in the synthesis of in vivo second messengers (phospholipids). Therefore, genes encoding proteins having this enzymatic activity may be used in controlling the signal transduction system.

Protein kinase:

Protein phosphatase:

Protein serine/threonine kinase:

Protein serine/threonine phosphatase:

Protein tyrosine kinase:

Protein tyrosine phosphatase:

Protein tyrosine/serine/threonine phosphatase:

Transmembrane receptor protein tyrosine kinase:

These enzymes are involved in the intracellular signal transduction occurring in plant biological process such as development and differentiation, and response to environmental factors. Therefore, genes encoding proteins having these enzyme activities are extremely important research subjects.

Sulfotransferase:

This enzyme is important for sulfur nutrition in plants. Therefore, genes encoding proteins having this enzymatic activity are useful in breeding plants responsive to nutrients and/or environment.

3′,5′-Cyclic-nucleotide phoshodiesterase (TOC1 homolog):

Dyneinalpha chain, flagellarouterarm (Adagio3 =FKFlhomolog):

These enzymes are involved in regulation of circadian rhythm in plants. Circadian rhythm refers to a biological rhythm having a period of approximately 24 hours. Therefore, genes encoding proteins having these enzyme activities are useful in breeding environment-responsive plants. Examples of full-length cDNA clones of the present invention to which these GO terms are assigned, include J013116P12 and J013023A04.

DNA helicase (DDM1=SYD homolog):

This enzyme controls DNA methylation. Therefore, genes encoding proteins having this enzyme activity are useful in breeding plants by controlling gene expression. An example of clones to which this GO term is assigned includes J013133N02.

Calpain:

This enzyme is involved in the control of starch storage in rice. Therefore, genes encoding proteins having this enzyme activity are useful in the improvement of phenotypes associated with storage substances in plants. Examples of clones to which this GO term is assigned include 002-108-E01 and J013167021.

Heat shock protein (HSP100 homolog):

This enzyme is an environmental stress-responsive enzyme. Therefore, genes encoding proteins having this enzyme activity areuseful inbreedingheat-tolerantplants. Examplesof clones to which this GO term is assigned include J023007C17 and 001-027-D01.

Ubiquitin activating enzyme:

Ubiquitin ligase:

These enzymes can be useful in elucidating the basic physiological regulatory mechanism of plants. This GO term is assigned to, for example, J033076H04 and 001-046-C03.

Histidine kinase (Wooden leg homolog):

HD-zipped (Revoluta homolog):

These enzymes are involved in intracellular signal transduction occurring in plant biological process such as development and differentiation, and response to environmental factors. Therefore, genes encoding proteins having these enzyme activities are extremely important researchsubjects. These GO terms are assigned to, for example, J013112K17 and 001-023-H08.

Receptor-like protein kinase (bril homolog):

Serine/threonine kinase (shaggy-like homolog):

These enzymes play important roles in signal transduction mediated by brassinosteroid, which is a phytohormone having important actions such as growth promotion, an increase in the plant yield, or enhancement of stress tolerance. Therefore, genes encoding proteins having these enzyme activities can be extremely important research subjects. These GO terms are assigned to, for example, J033069J12 and J033061L20.

Serine/threonine kinase (ERECTA homolog):

Transporter (shoot gravitropism 2 homolog):

Replication licensing factor (Prolifera homolog):

These enzymes control morphogenesis of plants. Therefore, genes encoding proteins having these enzyme activities can be extremely important research subjects. These GO terms are assigned to, for example, J033070P05, J013087B12 and J033041P20.

GO terms associated with the category “transporter” include proteins which are all involved in substance transport within plant bodies or between the inside and outside of plants. Substance transport in plants is important biologically and industrially. The GO terms associated with “transporter” and their industrial usefulness is specifically described below.

Ammonium transporter:

This protein functions as an ammonium ion transporter within plant bodies. Therefore, genes encoding proteins having this activity are useful in breeding plants focusing on nitrogen metabolism. Nitrogen absorption capability of plants can be enhanced by increasing expression of this protein.

Cobalt ion transporter:

Heavy metal ion transporter:

These proteins function as a heavy metal transporter within plant bodies. Therefore, genes encoding proteins having these activities are useful in breeding plants capable of growing on heavy metal-polluted soil. For example, plants become tolerant to heavy metal-polluted soil bysuppressing activities of these proteins. Plants and microorganisms in which these proteins are expressed can be utilized to recover the heavy metal-polluted soil.

Full-length cDNAs of the present invention were isolated fromavarietyof library sources. Comparisonof library sources from which cDNAs of the present invention are derived enables to detect genes specifically found in a specific library source. Flower organ-specific genes thus found based on such idea are shown in the “List of flower organ-derived clones” at the end of this specification.

Flower organs have functions that directly influence the rice yield such as flowering and fruition. Needless to say, genes specifically expressed in flower organs are industrially extremely useful in genetically breeding new rice plants. Furthermore, regions that control the expression of these genes may possibly control the expression of flower organ-specific genes. Therefore, the transcriptional regulatory region of thesegenes is useful, for example, in controlling the expression of proteins in seeds. Plant seeds contain many industrially important proteins such as enzymes influencing nutrition of seeds and potentially allergic proteins.

Regulation of these gene functions within plant bodies may change characteristics and morphology of plants. Examples of such changes are changes in salt and vermin tolerances, growth, and flowering time of plants, but not limited thereto.

Plant-derived DNAs of the present invention are not limited to those derived from rice, including DNAs derived from other plants as long as they have the function equivalent to that of the rice-derived DNA set forth in any of SEQ ID NOs: 1 through 28,469. Source plants are preferably monocot plants, more preferably poaceous plants, and most preferably rice. Whether a DNA has the function equivalent to that of rice-derived DNAs isolated by the present inventors or not can be judged by whether, compared to rice-derived DNAs, similar changes occur in plant bodies when said DNA is expressed in plants or when the function of said DNA is inhibited in plant bodies. DNAs which induce similar changes in plant bodies have the “function equivalent” to that of rice-derived DNAs isolated by the present inventors.

The present invention includes DNAs encoding proteins which are structurally analogous to a protein having the amino acid sequence set forth in any one of SEQ ID NOs: 28470 through 56791 and have functions equivalent to that of said protein. Such DNAs include, mutants, derivatives, alleles, variants and homologs of DNAs encoding proteins comprising amino acid sequences set forth in any of SEQ ID NOs: 28470 through 56791, in which one or more amino acid residues are substituted, deleted, added and/or inserted.

Examples of the method known to those skilled in the art for preparing DNAs encoding proteins whose amino acid sequences have been modified include the site-directed mutagenesis method (Kramer, W. & Fritz, H.-J., Methods Enzymol, 1987, 154: 350). Furthermore, mutation in the amino acid sequence of a protein due to the mutation in the coding nucleotide sequence may occur spontaneously. Thus, even DNAs encoding proteins having amino acid sequences in which one or more amino acids have been substituted, deleted, added and/or inserted are included in the DNAs of the present invention as long as they encode proteins having functions equivalent to those of the natural proteins (SEQ ID NOs: 28470 through 56791).

To maintain the original function of a protein, an amino acid of the protein is preferably substituted with an amino acid thathas the similar property as that of the amino acid of the protein. For example, amino acids belonging to the same group shown below have similar property. Even when an amino acid is substituted with another amino acid in the same group, the essential function of the protein does not change in most cases. Such amino acid substitution is referred to as conservative substitution, which is well-known modification of amino acid sequences without altering the original function of a protein.

Non-polar amino acids: Ala, Val, Leu, Ile, Pro, Met, Phe and Trp;

Non-charged amino acids: Gly, Ser, Thr, Cys, Tyr, Asn and Gln;

Acidic amino acids: Asp and Glu; and

Basic amino acids: Lys, Arg and His.

The number of amino acids that are mutated is not particularly restricted, as long as the a mutant protein is functionally equivalent to the original protein. Normally, it is within 50 amino acids, preferably within 30 amino acids, more preferably within 10 amino acids, and even more preferably within 3 amino acids. The site of mutation may be any site, as long as a mutant protein is functionally equivalent to the original protein.

DNAs having changes in their nucleotide sequences due to degeneracy are also included in the present invention. Degeneracy refers to a mutation in nucleotide sequences, which does not cause any mutation in amino acid residues in proteins.

Examples of other methods for preparing DNAs encoding proteins functionally equivalent to those comprising the amino acid sequences set forth in SEQ ID NOs: 28470 through 56791 are those using the hybridization technique (Southern, E. M., J. Mol. Biol., 1975, 98: 503) and polymerase chain reaction (PCR) technique (Saiki, R. K., et al., Science, 1985, 230:1350; Saiki. R. K., et al., Science, 1988, 239: 487). Using the nucleotide sequences of cDNAs of the present invention (SEQ ID NOs: 1 through 28469) or their portions as a probe, and oligonucleotides specifically hybridizing to these cDNAs as a primer, those skilled in the art would readily isolate DNAs highly homologous to these cDNAs from rice and other plants. Thus, DNAs of the present invention include DNAs encoding proteins having functions equivalent to those of the proteins comprising amino acid sequences set forth in SEQ ID NOs: 28470 through 56791 which can be isolated by the hybridization and PCR techniques.

For the isolation of such DNAs, hybridization is carried out preferably under the stringent condition. The stringent hybridization condition in the present invention refers to the condition of 6 M urea and 0.4% SDS in 0.5× SSC, or the stringent hybridization condition equivalent thereto. Under the more stringent condition, for example, that of 6 M urea and 0.4% SDS in 0.1× SSC, more highly homologous DNAs can be isolated. High homology refers to the sequence homology of at least 50% or more, preferably 70% or more, more preferably 90% or more, and most preferably 95% or more (e.g. 96, 97, 98 and 99%) in the entire amino acid sequence.

Preferably, isolated nucleic acid of the present invention includes a nucleotide sequence that is at least 50% identical to any one of the nucleotide sequence shown in SEQ ID NO: 1 to 28469. More preferably, the isolated nucleic acid molecule is at least 60%, 70%, 80%, 85%, 90%, 91%, 92%, 93%, 94%, 95%, 96%, 97%, 98%, 99%, or more, identical to anyone of the nucleotide sequence shown in SEQ ID NO: 1 to 28469.

Homologies of amino acid and nucleotide sequences (sequence identity) can be determined using algorithm BLAST reported by Carlin and Altschul (Proc. Natl. Acad. Sci. USA, 1990, 87: 2264-2268; Proc. Natl. Acad. Sci. USA, 1993, 90: 5873). Programs called BLASTN and BLASTX based on the BLAST algorithm have been developed (Altschul, S. F., et al., J. Mol. Biol., 1990, 215: 403). In the case of analyzing nucleotide sequences using BLASTN, parameters are set at, for example, score=100, and word length=12. Furthermore, in the case of analyzing amino acid sequences using BLASTX, parameters are set at, for example, score=100 and word length=12. In the case of using BLAST and Gapped BLAST programs, default parameters are used. Specific techniques of these analytical methods are well-known (see the NCBI website, http://www.ncbi.nlm.nih.gov/).

cDNAs of the present invention can be prepared by synthesizing cDNAs based on mRNAs extracted from plant such as rice, inserting them into a vector such as λ%ZAP to construct cDNA libraries, and developing them to perform the colony hybridization or plaque hybridization using probes comprising the entire nucleotide sequences set forth in SEQ IDNOs: 1 through 28469 or portions thereof as probes or to carry out PCR using primers designed based on the nucleotide sequences set forth in SEQ ID NOs: 1 through 28469.

Comparison of nucleotide sequences of cDNAs of the present invention with the genomic nucleotide sequence, can clarify the locations of the transcription initiation point or boundaries between exons and introns in the genome. The genome nucleotide sequence is available, for example, as the sequence of the BAC/PAC clone presented by the International Rice Genome Sequencing Project (IRGSP) . The nucleotide sequences of full-length cDNA clones of the present invention are aligned to map them to each exon of the genomic sequence. This mapping gives information as to at what positions of the genome transcription starts and ends, and from what sites introns are excised.

Full-length cDNA clones of the present invention can be used to perform comprehensive expression analysis as well as proteome analysis at the transcription level. For example, full-length cDNA clones of the present invention as they are or their regions that would be clone-specific, such as 5′-UTR or 3′-UTR region of the sequences of cDNA clones, may be immobilized on glass plates to prepare microarrays for expression analysis of rice. DNA fragments to be immobilized can be synthesized by known techniques such as PCR. Methods of immobilizing cDNA clones and their fragments on glass plates are also well-known in the art. cRNAs prepared from various cells of rice are allowed to hybridize to the microarrays thus obtained, thereby obtaining expression patterns of genes characteristic for each cell.

A DNA array is composed of a substrate onto which a large quantity of probes are attached in high density so as to analyze changes in the expression levels of a large amount of genes at high speed. The use of DNA arrays enables to comprehensively find out genes whose expression levels are altered in plant cells exposed to the specific condition. This analytical technique is referred to as gene expression profile analysis. One of factors that greatly influence usefulness of DNA arrays as an analytical tool, is the number of probes attached to DNA arrays. It may be easily understood that, for example, a DNA array comprehensively containing all of the genes of a plant is an ideal tool for the gene expression profile analysis. In fact, there is no higher plant in which all of the existing genes of which have been analyzed. Therefore, in reality, usefulness of DNA arrays depends on how as many genes as possible they contain. The present inventors determined the structures of a great number of rice-derived genes using a rice DNA array. It can thus be said that the present invention remarkably improved the usefulness of rice DNA arrays.

DNA arrays used in the present invention can contain probes comprising nucleotide sequences specifically found in each of 28,469 clones. The probes constituting DNA arrays of the present invention may include probes of any clones selected from28,469 clones. The number of selected clones is, for example, 10% or more, usually 30%, preferably 50% or more, more preferably 70% or more, and most preferably 80% or more of 28,649 clones. The more the number of selected clones is, the more genes are comprehensively analyzed.

The proteome analysis can be carried out, for example, as follows. First, the coding regions of cDNAs excised from the full-length cDNA clones are each linked to an appropriate vector to express their proteins using Escherichia coli and yeast systems. Proteins thus obtained are purified, and can be used in structural analysis, interaction analysis, complementation test for mutation, etc.

DNAs of the present invention can be used to produce mutant plants. Mutant plants expressing DNAs of the present invention can be produced by inserting the above-described DNA into an appropriate vector, introducing the vector with the insertion into plant cells by methods described below, and regenerating transformed plant cells thus obtained. On the other hand, plants in which the expression of DNAs of the present invention is suppressed can be produced, for example, as described below, by inserting a DNA that suppresses the expression of a DNA of the present invention into an appropriate vector, transducing the vector with the insertion into plant cells, and regenerating the transformed plant cells thus obtained. Herein, the “suppression of DNA expression” includes the suppression of transcription of DNAs as well as their translation into proteins. Furthermore, it also includes not only complete block but also reduction of DNA expression, and furthermore, inhibition of the translated protein to express its inherent function within plant cells.

The antisense technique is the most common method for suppressing the expression of a specific endogenous gene in plants employed by those skilled in the art. Antisense effects in plant cells were proved for the first time by Ecker et al. who demonstrated antisense effects of the antisense RNA transduced into plant cells by electroporation (Ecker, J. R. & Davis, R. W, Proc. Natl. Acad. Sci. USA, 1986, 83: 5372). Thereafter, a report presenteda decrease in the target gene expression in tobacco and petunia plants due to the antisense RNA expression (van der Krol, A. R., et al., Nature, 1988, 333: 866), and, to-date, the antisense technique has been established as a means of inhibiting gene expression in plants.

For example, antisense nucleic acids inhibit the expression of a target gene, in the following manners.

Inhibition of transcription initiation due to triple helix formation;

Transcriptional inhibition due to hybridization to the site of opened loop structure locally formed by RNA polymerase;

Transcriptional inhibition due to hybridization to RNA whose synthesis is in progress;

Inhibition of splicing due to hybridization to the boundaries of introns and exons;

Inhibition of splicing due to hybridizaation to the site of spliceosome formation;

Inhibition of transition of mRNA from the nucleus to cytosol due to hybridization to the mRNA;

Inhibition of splicing due to hybridization to the capping site and poly(A) addition site;

Inhibition of translational initiation due to hybridization to the translation initiation factor binding site;

Inhibition of translation due to hybridization to the ribosome binding site near the initiation codon;

Inhibition of peptide chain elongation due to hybridization to the mRNA coding region and polysome binding site; and

Inhibition of gene expression due to hybridization to the interaction site between nucleic acid and protein.

Thus, antisense nucleic acids inhibit various processes such as transcription, splicing, and translation to suppress the expression of a target gene (Hirashima and Inoue, “New Experimental Biochemistry Manual 2, Nucleic Acid IV, Gene Duplication and Expression,” The Japanese Biochemical Society, ed., Tokyo Kagaku Dojin, 1993, pp. 319-347).

Antisense sequences used in the present invention may inhibit the expression of a target gene by any of the above-described actions. As an embodiment, an antisense sequence designed so as to be complementary to the non-coding region near the 5′-end of mRNA of a target gene would inhibit its translation. Sequences complementary to the coding region or non-coding region of the 3′-side can also be used. Antisense DNAs used in the present invention include DNAs comprising antisense sequences of not only the coding region but also non-coding region of a target gene.

Antisense DNA to be used is linked downstream of an appropriate promoter and the transcription termination signal is preferably linked to the 3′-end of antisense DNA. Antisense DNA thus prepared can be transformed to a desired plant by known methods. Although sequences of antisense DNAs are preferably complementary to the endogenous gene of a plant to be transformed or a portion thereof, they may not be completely complementary thereto as long as they are capable of efficiently inhibit target gene expression. The transcribed RNAs are preferably 90% or more and most preferably 95% or more complementary to the transcript of a target gene. For the effective inhibition of target gene expression, an antisense sequence has at least 15 nucleotides or more, preferably 100 nucleotides or more, and more preferably 500 nucleotides or more. Antisense DNAs commonly used are less than 5 kb, preferably less than 2.5 kb.

It is also possible to inhibit the expression of endogenous genes using a DNA encoding ribozyme. Ribozyme refers to an RNA molecule with catalytic activity. Various activities of ribozymes are known, and, among them, researches focusing on the ribozyme activity to cleave RNA have enabled to design ribozymes that site-specifically cleave RNA. Large-sized ribozymes such as group I intron type RNA and M1 RNA included in RNase P, consist of 400 or more nucleotides, while some ribozymes have about 40 nucleotides of the active domain, including hammerhead and hairpin ribozymes (Makoto Koizumi and Eiko Ohotsuka, Protein Nucleic Acid Enzyme, 1990, 35: 2191).

For example, the self-splicing domain of hammerhead ribozymes cleaves the 3′-side of C15 in the G13U14C15 sequence. Base pairing of U14 with A9 is thought to be important for that cleaving activity, and it has been demonstrated that said domain can be cleaved even when C15 is replaced by A15 or U15 (Koizumi, M., et al., FEBS Lett., 1988, 228: 228). Ribozymes whose substrate-binding site is complementary to an RNA sequence near the target site, recognize a sequence such as UC, UU or UA in the target RNA and cleave it like restriction enzymes (Koizumi, M., etal., FEBS Lett., 1988, 239: 285; Koizumi, M. and Ohotsuka, E., Protein Nucleic Acid Enzyme, 1990, 35: 2191; Koizumi, M., et al., Nucl. Acids Res., 1989, 17: 7059). For example, DNAs of the present invention (SEQ ID NOs: 1 through 28469) contains several potential ribozyme target sites.

Hairpin ribozymes, which are found, for example, in the negative strand of satellite RNA of tobacco ring spot virus (Buzayan, J. M., Nature, 1986, 323: 349), are also useful for the purpose of the present invention. It was shown that hairpin ribozymes can also be designed to cleave RNA target site-specifically (Kikuchi, Y. and Sasaki, N., Nucl. Acids Res., 1991, 19: 6751; Kikuchi, Y., Chemistry and Biology, 1992, 30: 112).

A ribozyme designed to cleave a target site is linked to a promoter such as 35S promoter of cauliflower mosaic virus and the transcription termination sequence so as to be transcribed in plant cells. However, when an excessive sequence is attached to the 5′- and 3′-ends of the transcribed RNA, the ribozyme activity might be sometimes lost. In such a case, it is possible to arrange a different cis-acting trimming ribozyme on the 5′- and 3′-ends of the ribozyme portion so as to accurately excise only the ribozyme portion from the transcribed RNA containing said ribozyme (Taira, K. et al. , Protein Eng. , 1990, 3: 733; Dzianott, A. M. and Bujarski, J. J., Proc. Natl. Acad. Sci. USA, 1989, 86: 4823; Grosshans, C. A. and Cech, T. R., Nucl. Acids Res., 1991, 19: 3875; Taira, K. et al., Nucl. Acids Res., 1991, 19: 5125). Furthermore, it is also possible to arrange these constitutive units in tandem so as to cleave at plural sites within the target gene, thereby enhancing its effect (Yuyama, N. et al., Biochem. Biophys. Res. Commun., 1992, 186: 1271). Expression of a target gene of the present invention can be suppressed by specifically cleaving the transcript of the target gene.

Endogenous gene expression can be suppressed by RNA interference (RNAi) using a double-stranded RNA having a sequence identical or analogous to a target gene sequence. RNAi refers to the phenomenon wherein introduction of a double-stranded RNA, which comprises a sequence identical or analogous to a target gene sequence, into cells suppresses expression of both the transgene and the target endogenous gene. Although the mechanism of RNAi has not been elucidated in detail, it is thought that the double-stranded RNA introduced is decomposed into small fragments, which, in turn, become an indicator of the target gene by some meansand induce degradation of the target gene. It is also known that RNAi is effective in plants (Chuang, C. F. & Meyerowitz, E. M., Proc. Natl. Acad. Sci. USA, 2000, 97: 4985). For example, for the suppression of expression of DNA encoding the target protein in plants, DNA encoding said protein or a double-stranded RNA having the sequence analogous thereto may be introduced into a plant to select, out of the plant bodies thus obtained, plants in which the expression level of said protein is reduced compared to the wild type plant. Genes used in RNAi are not necessarily completely identical to a target gene, but have at least 70% or more, preferably 80% or more, more preferably 90% or more, and most preferably 95% or more of sequence identity to the target gene. Sequence identity can be determined by the above-described technique.

Expression of an endogenous gene can also be inhibited by co-suppression due to transformation with DNA having a sequence identical or analogous to a target gene. “Co-suppression” refers to the phenomenon in which introduction of a gene having a sequence identical or analogous to a target endogenous gene into plants by transformation, inhibits the expression of both of the transgene and the target endogenous gene. Although the mechanism of co-suppression has not been clarified in detail, it is thought to at least partially overlap that of RNAi. This phenomenon has been observed also in plants (Smyth, D. R., Curr. Biol., 1997, 7: R793; Martienssen. R., Curr. Biol., 1996, 6: 810). For example, a plant body having co-suppressed DNA encoding a protein can be obtained by preparing a vector DNA that expresses DNA encoding said protein or DNA having the sequence analogous thereto, transforming a target plant with the vector, and selecting plants in which the expression level of said protein is reduced compared to the wild type plant body. Although a gene used in co-suppression needs not be completely identical to a target gene, it has at least 70% or more, preferably 80% or more, more preferably 90% or more, and most preferably 95% or more sequence identity to the target gene. Sequence identity can be determined using the aforementioned technique.

The present invention provides a method of producing a transformed plant body comprising the steps of introducing a DNA of this invention into plant cells and regenerating plant bodies from said plant cells.

In the present invention, there is no particular limitation in the types of plants from which plant cells are derived. There is also no particular limitation in the types of vectors used in the transformation of plant cells, as long as they are capable of expressing the transgene in said cells. For example, a vector to be used has a promoter (such as 35S promoter of cauliflower mosaic virus) that constantly express a gene in plant cells and is inducible by an extraneous stress. Herein, “plant cells” include plant cells in various forms such as suspended cultured cells, protoplasts, leaf sections, and calli.

The vectors canbe introduced into plant cells using various methods known to those skilled in the art such as the polyethylene glycol method, electroporation method, method mediated by Agrobacterium, and particle gun method. In the method mediated by Agrobacteriun (e.g., EHA101), it is possible to use, for example, the ultrahigh speed monocot transformation method (Japanese Patent No. 3141084). Furthermore, the particle gun method can be carried out by using, for example, the product from BioRad. Regeneration of plant bodies from the transformed plant cells may be conducted using methods known to those skilled in the art depending on the type of plant cells (Toki, S., et al., Plant Physiol., 1995, 100: 1503).

Several methods of producing transformed rice plant bodies have already been established and extensively used in the technical field to which the present invention pertains. These methods include the method of introducing a gene into protoplasts using polyethylene glycol to regenerate plant bodies (suitable for Oryza sativa L ssp. Indica) (Datta, S. K., “In Gene Transfer To Plants,” Potrykus, I. and Spangenberg, Eds. , 1995, pp. 66-74); Toki, S., et al., “the method of introducing a gene into protoplasts with the electric pulse to regenerate plant bodies (suitable for Oryza sativa L ssp. japonica), ” Plant Physiol., 1992, 100: 1503); the method of directly introducing a gene into cells by the particle gun technique to regenerate plant bodies (Christou, P., et al., Biotechnology, 1991, 9: 957), and the method of introducing a gene into cells mediated by Agrobacterium to regenerate plant bodies (Hiei, Y., et al., Plant J., 1994, 6: 271). These methods can be preferably used in the present invention.

Once the transformed plant body in which a DNA of the present invention has been introduced is obtained, it is possible to obtain progenies from said plant by sexual or asexual reproduction. It is also possible to obtain propagation materials from said plant body and its progeny or clone, which allows mass-production of said plant body. Propagation materials which can be thus obtained are included in the present invention. Propagation materials of the present invention include all of the materials capable of regenerating the plant bodies having the genetic characteristics introduced into the transformed plant body of this invention, for example, seeds, fruits, spikes, tubers, tuberous roots, stubs, calli, and protoplasts.

The present invention also relates to proteins having the amino acid sequences encoded by the above-described nucleic acids. Proteins of this invention can be prepared by the gene recombination technique using the aforementioned nucleic acids. Recombinant proteins of this invention can be prepared by inserting a DNA encoding a protein of this invention into an appropriate vector, introducing said vector into suitable cells, culturing transformed cells thus obtained, and purifying the expressed protein.

A recombinant protein can be expressed as a fusion protein with another protein to facilitate its purification and detection. For example, a method for expressing in a host Escherichia coli a protein as a fusion protein with a fusion partner protein as described below. Preferable vectors for each fusion protein are parenthetically shown.

Maltose bindingprotein (vector PMAL series, New England BioLabs, USA),

Glutathion-S-transferase (GST) (vector pGEX series, Amersham Pharmacia Biotech), and

Histidine tag (pET series, Novagen)

There is no particular limitation in the type of host cells to be used for producing recombinant proteins as long as they are suitable for expressing the recombinant proteins. Besides the aforementioned Escherichia coli, for example, host cells such as yeasts, various animal and plant cells, and insect cells may be used. The vector can be introduced into cells by various methods known to those skilled in the art. For example, introduction into Escherichia coli can be performed by the method using calcium ion (Mandel, M. & Higa, A., Journal of Molecular Biology, 1970, 53, 158-162; Hanahan, D., Journal of Molecular Biology, 1983, 166, 557-580). Recombinant proteins expressed in host cells can be recovered and purified from the host cells or their culture supernatant by the method known to those skilled in the art. When recombinant proteins are expressed as a fusion protein with the above-described fusion partner such as maltose-binding protein, the proteins of interest can be easily purified by affinity chromatography.

For more easily performing the affinity purification, an amino acid sequence recognized by protease may be inserted into the fusion protein. For example, amino acid sequences of the maltose-binding protein and a protein to be produced are connected mediated by a protease recognition sequence. After capturing the fusion protein using the action of maltose-binding protein, the target protein can be recovered by the action of the protease. In this case, any protease not acting on the target protein may be used.

The recombinant protein thus obtained can be used to prepare antibodies binding thereto. For example, polyclonal antibodies can be obtained by immunizing animals such as rabbits with a purified protein of the present invention or its partial peptide. After confirming the titer elevation of immunized animals, the blood is withdrawn, and the serum is recovered to obtain anti serum against the target protein. Alternatively, polyclonal antibodies can be obtained by purifying IgG from the antiserum. Furthermore, purified antibodies can be obtained by carrying out immuno-affinity purification using the target protein as an antigen.

Furthermore, monoclonal antibodies can be prepared by fusing myeloma cells and antibody-producing cells of the animal immunized with the above-described protein or peptide, isolating monoclonal cells (hybridoma) producing the target antibodies, and obtaining the antibodies from said cells. The antibodies thus obtained can be used in the purification and detection of proteins of this invention. The present invention includes antibodies capable of binding to proteins of this invention.

The present invention also provides a database including information on the nucleotide sequences of rice full-length cDNA clones ofthis invention and/or information on their amino acid sequences. A data base refers to a collection of information on the nucleotide sequences and/or amino acid sequences contained as retrievable and machine-readable data. Databases of the present invention contain at least one of the nucleotide sequences of rice full-length cDNAs of this invention. Databases of the present invention may consist of rice full-length cDNAs of this invention, or include information on nucleotide sequences of known full-length cDNAs, ESTs, etc. In databases of the present invention, not only information on the nucleotide sequences but also information related thereto such as the gene function revealed by this invention and names of clones retaining those full-length cDNAs may be recorded together or linked thereto.

Databases of the present invention are useful for acquiring a full-length gene based on information on gene fragments. Databases based on this invention all comprise information on full-length cDNA nucleotide sequences. Therefore, by comparing nucleotide sequences of gene fragments obtained by the gene expression analyses using a DNA array and subtraction method with the information of this database, the full-length nucleotide sequences of genes can be revealed.

Furthermore, since databases of the present invention contains information associated with rice genes, it is useful for isolating rice homologs based on information on nucleotide sequences of genes isolated from other species.

At present, gene expression analysis such as DNA array analysis enables to obtain information on diverse gene fragments. These gene fragments, ingeneral, are used as a tool for obtaining their full-length sequence. When a gene fragment is derived from a known gene, its full-length can be easily elucidated by comparing it with a known database. However, when no identical nucleotide sequence is found in a known database, cloning of gene fragments must be carried out to obtain the full-length cDNA. It is often difficult to obtain full-length nucleotide sequences based on information on DNA fragments. Without obtaining a full-length gene, it is impossible to deduce the amino acid sequence of a protein encoded by that gene. Databases of the present invention would contribute to identification of full-length cDNAs corresponding to gene fragments that cannot be elucidated by the already known gene databases.

Databases of the present invention can also be used to isolate genes associated with diverse characteristics. For example, the relationship between polymorphism markers and phenotypes of organisms has been clarified. cDNA nucleotide sequences of this invention are mapped to genomic DNA and compareded with the information on polymorphism markers whose association with phenotypes is known. A gene having a polymorphism marker within the transcriptional regulatory region and its exonswould associate with the phenotype correlated to the polymorphism marker. Thus, the use of information on nucleotide sequences of cDNAs of this invention enables positional cloning in silico. The polymorphism markers usable in such analysis include, for example, SNPs. SNPs may be examined for the relationship with a single polymorphism site, or focused on a plurality of SNPs.

Furthermore, the present invention relates to a method of obtaining the transcriptional regulatory region of rice, wherein said method comprising the following steps:

(1) mapping the nucleotide sequence set forth in any of SEQ ID NOs: 1 through 28469 to the genomic nucleotide sequence of rice, and

(2) judging the region containing the transcriptional regulatory region found upstream of the 5′ most end of the mapped region as the transcriptional regulatory region of the gene mapped in the step (1).

Gene expression is controlled by transcription factors. In genomes, the region containing the nucleotide sequence recognized by a transcription factor is referred to as the transcription regulatory region, which usually exists in the region on the 5′-side of the transcription initiation point. For example, the region comprising several hundreds to several kbs of DNAs arranged in tandem often has the transcriptional regulatory action. In genomes, in contrast to a nucleotide sequence encoding mRNA which is divided by many introns, the transcriptional regulatory region exists with being linked in tandem. Therefore, once the transcription initiation point can be mapped to the genome, the transcriptional regulatory region can be relatively easily obtained. The method of obtaining the transcriptional regulatory region based on information on nucleotide sequences of the full-length cDNAs, will be described below in more detail.

First, of the nucleotide sequences of full-length cDNAs, sequences containing the 5′-end, in particular, are mapped to the genomic sequence. Genomic nucleotide sequences of any species may be used, and the genome of Oryza sativa L. ssp. japonica is preferably used. Mapping reveals the position coinciding with the 5′-end as the transcription initiation point. Nucleotide sequences near the transcription initiation point will be the analytical subject as the candidate sequence. The candidate sequence is usually selected from the region ranging from (−) several kb to (+) several hundreds b counting the transcription initiation point to be “1.” Herein, the minus (−) numerals refer to the values counted in the direction toward the 5′-end, while the plus (+) numerals the values counted in the direction toward the 3′-end. More specifically, the candidate sequence may be in the region of from -2 kb to +500 b, for example, from −1 kb to +200 b, and preferably from −1 kb to +1 b. The transcriptional regulatory region can be predicted by searching for the transcription factor-binding consensus sequence. Nucleotide sequences found in genomes capable of binding to the transcription factor are referred to as cis sequences. For example, TATA box is a representative cis sequence. Many nucleotide sequences have been identified in the transcription factor-binding consensus sequence, and information on a specific sequence can be obtained, for example, from the transcription factor-binding consensus sequence database TRANSFAC (http://transfac.gbf.de/homepage/databases/transfac/transf ac.html).

Analytical results determine the region containing the cis sequence in candidate sequences as the transcriptional regulatory region. Further analysis can be done based on their interaction with the transcription factor recognizing cis sequence constituting said transcriptional regulatory region. For example, the footprinting method and gel shift assay have been used as the analytical methods for clarifying the relationship between the transcriptional regulatory region and transcription factor. These analytical methods identify a region necessary for the transcription in the transcriptional regulatory region thus selected. Furthermore, a reporter assay can be performed to assess the transcriptional action of the transcriptional regulatory region obtained by the present invention.

Full-length cDNAs of the present invention would contain the nucleotide sequence of the 5′-end of mRNA, and are useful as a tool for obtaining the transcriptional regulatory region. Depending on purposes, A cDNA derived from a special library source can also be used to obtain the transcriptional regulatory region. For example, flower bud-specific cDNAs can be used to obtain the transcriptional regulatory region specific to flower bud. Furthermore, cDNAs obtained from tissues exposed to various stresses can be used to obtain the transcriptional regulatory region associated with stress. As described above, the genome drafts of indicassp. (Yu, J. et al., “A draft sequence of the rice genome (Oryza sativa L. ssp. indica)” Science, 2002, 296, 79-92) and japonica ssp. (Goff, S. A. et al., “A draft sequence of the rice genome (Oryza sativa L. ssp. japonica)” Science, 2002, 296, 92-100) , have been already published. Therefore, information on sequences of cDNAs of this invention can be used to obtain many transcriptional regulatory regions.

The present invention provides and characterizes full-length cDNAs comprehensively. These full-length cDNA scan be used for annotation of correct gene coding region, determination of exons and introns, comprehensive expression analysis on the transcriptional level, and proteome analysis. Additionally, they are useful in producing plants having the different characteristic from the wild type due to the inhibition of their expression and function within the plant bodies.

Since cDNAs of the present invention have a strong probability of being full-length, information on these nucleotide sequences would efficiently predict the transcriptional regulatory region. To obtain the transcriptional regulatory region based on the gene nucleotide sequences, the transcriptional initiation point should be correctly determined, which can be successfully achieved using information on nucleotide sequences of full-length cDNAs whose 5′-end nucleotide sequences are completely provided. Any patents, patent applications, and publications cited herein are incorporated by reference in their entirety.

The present invention is explained in more detail with reference to examples, but is not be construed as being limited thereto.

### EXAMPLE 1

Acquisition of cDNA Clones

(1) Starting Materials and Method For Constituting a Full-Length cDNA Library

The rice genome project of Japan has obtained EST clones from various tissues and organs at each developmental stage ofrice. However, most of them were derived from non-full-length cDNA libraries. ESTs are effective in cataloging the expressed genes, but not suitable for functional genome analysis. Starting materials for the library construction are extremely important to collect as many types of clones as possible. Table 1 is the list of starting materials for constructing a library suitable for functional genome analysis.

TABLE 1

| | |
|---|---|
| Library | Starting materials for Full-length |
| No | cDNA library construction |

Seedlings two weeks
after germination

| | | |
|---|---|---|
| 1 | Normally grown | Green Shoot |
| 2 | Normally grown | Root |
| 3 | Dark grown | Etiolated Shoot |
| | (Radiation and Oxidative) | |
| 4 | +UVB | 550 J/m² |

Calli ten days after transfer to new medium

| | | |
|---|---|---|
| 5 | Normally grown | |
| | (Temperature) | |
| 6 | +cold | cold treated (at 6° C.) |
| 7 | +heat | heat treated (at 45° C.) |
| | (Hormone) | |
| 8 | +auxin | 2 ppm of NAA |
| 9 | +Cytokinine | 2 ppm of BAP |
| 10 | +ABA | 2 ppm of ABA |
| | (Chemical) | |
| 11 | +Cd | 6 ppm of CdCl₂ |

Germinating seeds three days after imbibition

| | | |
|---|---|---|
| 12 | Normally grown | |
| | (Hormone) | |
| 13 | +auxin | 2 ppm of NAA |
| 14 | +Cytokinine | 2 ppm of BAP |
| | Panicles | |
| 15 | less than 1 cm stage | |
| 16 | less than 5 cm stage | |
| 17 | more than 5 cm stage | |
| 18 | one day after flowering | |
| 19 | two weeks after flowering | |
| 20 | three weeks after flowering | |

To date, the Foundation of Advancement of International Science (FAIS) has constructed 20 types of rice libraries enriched with full-length cDNAs by using the oligo-capping method combined with normalization and size fractionation. To avoid PCR bias, the number of amplification cycles during the construction of these libraries was minimized. Furthermore, about a half of the resulting cDNA were cloned into a vector, which is part of the Gateway system, to facilitate high-throughput analysis of rice proteomics.

RIKEN has constructed 4 types of rice full-length cDNA libraries (shoots of seedlings, roots, calli and germinating seeds) by the RIKEN's original technologies which combined used the biotinylated CAP trapper method, thermo activation of reverse transcriptase by trehalose, normalization, the oligolinker method, the poly-stretch-less method, and the vector designed for the preferential cloning of long inserts.

Briefly, mRNAs were extracted by using the modified CTAB method, underwent cDNA synthesis, CAP trapping and normalization, and were cloned into the lambda vector. Lambda cDNA libraries were bulk-excised to plasmid libraries.

(2) Grouping of Terminal Sequences and Determination of Full-Length Sequences

Clones were randomly picked up from each library, and both the 5′- and 3′-ends of each clone were sequenced once. 175,642 of these clones were sequenced fromthe 3′-end, and 91,425 clones from the 5′-end. These clones (175,642) were clustered into 28,469 (nonredundant) groups by means of a grouping program using the nucleotide sequences determined from the 3′-end. All clones from each group were completely sequenced using the two methods (primer walking and shotgun methods). Assessment of sequence fidelity with the Fred value indicated that all representative 28,469 clones were sequenced with 99.98% fidelity. The length of the insert cDNA varied from 55 to 6528 bp, and its average length was 1655.0 bp.

### EXAMPLE 2

Functional Classification of Full-Length cDNA Clones

(1) BLAST Search

Sequence homology search by BLAST was conducted asfollows. Sequence data from 10 divisions of NCBI's GenBank (as of June 15, 2002; release version 130) were downloaded, and searches were carried out using BLAST N and BLAST X programs using 28,469 sequences as query (2002/7/20). Search subjects are the following 10 divisions: PRI, ROD, MAM, VRT, INV, PLN, BCT, VRL, PHG, and PAT. Sizes of searched database were as follows:

BLASTN: 1,212,780 sequences : 1,998,000,464 Letters

BLASTX: 623,580 sequences : 327,145,996 Letters

Alignment pattern was checked for sequence homology, and a similarity threshold of E<10⁻¹⁰ was used. Because of BLAST N search, 2603 cDNA clones were identical to already-registered rice genes, and classified into the identical rice genes. As a result ofBLAST X search, 5,607 clones were homologs of already known rice genes, 12,527 clones were homologous to already-known genes of plants other than rice, and 859 clones were homologous to already-known genes in organisms other than plants. These search results were shown in Table 2. In total, these homology searches enabled to assign potential functions to 21,596 (75.86%) clones.

“Results of BLAST search” at the end of this specification shows data of the highest homology to each clone obtained by the homology search of 28,469 representative full-length cDNA clones using BLAST N and BLSST X programs.

28,469 full-length cDNA clones were mapped to three types of already known rice genome sequence data, that is, the indica (aforementioned) and japonica (aforementioned) draft genomes. Sequence data of the gene coding regions are said to be very similar between japonica and indica subspecies. 94% of the 28,469 full-length cDNA clones were mapped to these rice draft genomic sequences. Mapping results are shown in Table 2.

TABLE 2

| | | | | | Number of |
|---|---|---|---|---|---|
| | | | Number of | | non-redundant |
| Genome | | Size | mapped | | Transcription |
| Sequence | Source | (Mbp) | clones | % | Unit |
| japonica draft | Ref 2 | 390 | 26930 | 94.6 | 18933 |
| genome | | | | | |
| indica draft | Ref 1 | 363 | 26784 | 94.1 | 19036 |
| genome | | | | | |
| BAC/PAC | IRGSP | 368 | 22162 | 77.8 | 15523 |
| clones from | | | | | |
| japonica | | | | | |

The 28,469 clones originated from 19,000 TU of the rice genome draft sequences using 94% of genome coverage data. Since, in this mapping method, paralog, pseudogene, and such are counted into a single orthologue gene, the actual TU number is expected to be larger. In fact, mapping results of the full-length cDNA clones to the BAC/PAC clone cluster derived from Chromosome 1 revealed that about 3,800 clones were mapped to 7,700 sites, indicating that approximately twice the cDNAs overlap in the rice gene.

Herein, “one transcription unit” means a group comprising a plurality of transcripts sharing exons. When two cDNAs are mapped to the same genomic region, an intron of one cDNA may have an exon of the other. In this case, the exon is not shared between cDNAs so that these cDNAs constitute different transcription units. In another case where two cDNAs are mapped to the same genomic region, these cDNAs are sometimes different in their orientations; an exon of one cDNA is mapped to the nucleotide sequence of an antisense strand of the other. In addition, in this case, since the exon is not shared, they constitute different transcription units.

Considering the genome coverage of less than 100% and the clustering results at the clone level, a unique population of 20,259 representative full-length CDNA clones is selected to hereafter discuss sequence data of total clones (28,469) and unique representative clones (20,259). A unique population refers to a cluster of cDNAs predicted to be transcribed from the same TU. A particular clone of the unique population representing said unique population is referred to as a representative clone.

The nucleotide sequence identity between CDSs predicted in the indica genome and ORFs in cDNAs of the present invention is 93%, while the amino acid identity is only 53%. These results may reflect the following reasons.

▪The longest ORF of cDNA of the present invention is not correct; and/or

▪the predicted CDS of indica genome is not correct.

From the results of InterPro search and such as described below, cDNA nucleotide sequences of the present invention would have higher fidelity than CDS predicted in indica, indicating that the actual acquisition of cDNAs improves the quality of information on gene sequence.

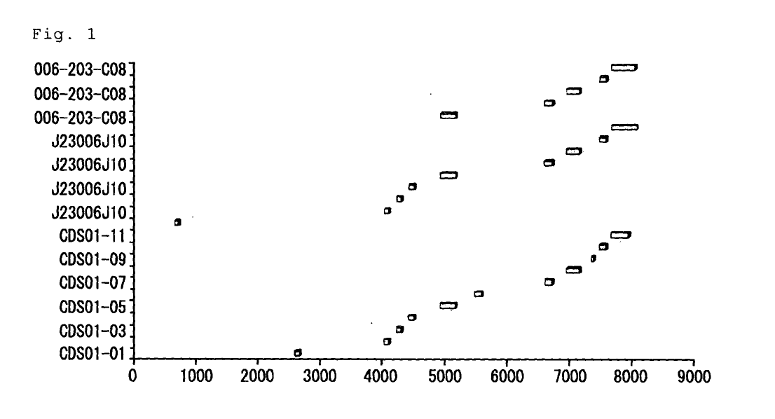

For example, comparison between predicted CDSs reported for the BAC clone derived from Chromosome 1 and mapping results of full-length cDNA clones of the present invention clearly revealed the presence of various differences between them. Results of comparison of them at the exon and intron levels are shown in FIG. 1. Results of comparison of cDNAs with all CDSs of Chromosome 1 are listed in Table 3.

TABLE 3

| | |
|---|---|
| Total number of BAC/PAC clones from | 418 (redundant) |
| Chromosome 1 | |
| Total number of annotated BAC/PAC | 387 (redundant) |
| clones from Chromosome 1 | |
| Total number of CDS | 9828 (redundant) |
| Total number of Transcription Units | 7,763 |
| mapped by FL-cDNA clones | by 3895 FL-cDNA clones |
| Total number of the hit CDS by | 4874 (49.6%) |
| FL-cDNA clones | by 3239 FL-cDNA clones |

In Table 3, 418 CDSs derived from Chromosome 1 are registered, 387 clones of which are annotated with CDS. Total number of CDS is 9828. On the other hand, when full-length cDNA clones of the present invention were mapped to the same BAC/PAC clones, the total number of TU mapped by 3895 full-length cDNA clones was 7763. Then, the coincidence of the mapped regions with the CDS regions was examined. When the coincidence of at least 10 bp in the same direction is judged as a coincidence, the total number of the CDSs that hit by mapped cDNA clones was only 4874, accounting for only 49.6% of the total CDS. That is, the results of assessing the predicted CDS based on the full-length cDNA sequences actually isolated from rice indicated that current programs cannot find CDS with sufficient accuracy, and that information on the cDNA nucleotide sequence isolated from the living body is important.

3) Changes in the Transcript Form

Of the 18,933 TUs mapped to the japonica genome draft, 5045 are multiexon TU that contains two or more transcripts with a plurality of forms. These transcripts were analyzed in detail focusing on the following conditions:

Difference in length of the 5′- and 3′-ends;

presence or absence of exons; and

initiation and termination sites of intron at the time of splicing.

As a result, of the 5045 TU, 2471 TU (13.1%) contains clones having variations in the above-described conditions. Contents of variations were as described below.

Difference in the 5′-end: 1673 TU (8,8%),

Difference in the 3′-end: 853 TU (0.5%),

Alternatively spliced exons: 94 TU (4.5%),

Difference in the intron initiation point: 180 TU (1.0%), and

Difference in the intron termination point: 241 TU (1.3%).

When the above TUs are simply summed up, the sum does not become 2471 because the identical TUs might have different combinations. Even considering the collection bias, the alternative splicing event is not frequent in rice.

Due to these differences in the transcript structure, alterations at the amino acid level accompanying with ORF changes among transcripts occurred at 1937 TU (78.4%) loci. In contrast, 902 antisense transcript pairs were found as the clones that were mapped in the opposite direction to the same genomic DNA region. The number of such clones was 1443.

4) Full-Length cDNAs Are Useful in the Promoter Analysis.

The expression analysis using a microarray system containing 8987 rice EST clones found that UV irradiation to rice led to the transcriptional upregulation of 58 EST clones. After confirming these upregulations by the real-time PCR method, candidate genes whose expression was enhanced were PR-10b and PBZl genes.

The full-length cDNA clones found to correspond to these ESTs were mapped to the region 10 kb away in the same BAC sequence derived from Chromosome 12. Furthermore, the use of sequences 1 kb upstream of the respective transcription initiation points of the full-length cDNA clones as queries for the cis element database (PLACE) fou nd cis elements such as GT1CONSENSUS and TBOXATGAPB. Although further experiments are needed to confirm whether these cis elements are actually involved in UV-related transcriptional upregulation or not, the use of information on full-length CDNA clones, as described above, will facilitate the promoter analysis of target genes from EST.

5) Protein Informatics Analysis

Amino acid sequences of proteins encoded by nucleotide sequences of cDNAs were deduced from the longest ORF (from ATG to term codon). Because of homology search, clones clearly encoding RNA (24,397 clones) , and clones with an ORF shorter than 100 amino-acid residues were excluded from analysis. 28,332 clones had ORF, and the average number of amino acid residues was 331 residues. Average numbers of nucleotides of the 5′- and 3′-untranslated region (UTR) were 259.83 and 398.41, respectively. 24,507 clones had ORFs exceeding 100 amino-acid residues.

6) Assessment of Completeness of the Full-Length cDNAs

cDNAs of the present invention were acquired using the CAP trapper method that is advantageous for acquiring full-length cDNAs. Whether cDNA clones of the present invention were full-length was confirmed by the following analyses.

Comparison of the full-length cDNA clones with the previously registered 859 cDNA sequences according to single-pass sequence data, revealed for 621 of 667 5′-end sequences and 570 of 648 3′-end sequences, the full-length cDNA clones of the present invention were longer than the registered cDNA sequence. Using full sequences of the clones, the full-length cDNA clones of this invention were longer than the registered cDNA sequence in 468 of 579 cases. Thus, it was obvious that most of the full-length cDNA clones of the present invention, which were found to coincide with the known nucleotide sequences, comprise longer terminal nucleotide sequences compared to the known sequences. Therefore, cDNAs of the present invention were confirmed to be a cluster of cDNAs that are likely to be full-length.

Furthermore, whether cDNAs have full-length or not can be confirmed if they contain the initiation and termination codons. More preferable full-length cDNA clones according to this invention may contain more 5′-UTRs of their transcript. Containg of all of the 5′-UTRs is not the essential prerequisite for the full-length cDNA. However, if transcript nucleotide sequences are maintained as completely as possible, the usefulness of full-length cDNAs increases. For example, when the transcriptional regulatory region is obtained, the transcription initiation point must be correctly determined for accurately obtaining it. For example, because of the alignment of cDNA clones and EST, etc., clones having a long nucleotide sequence on the 5′ side is likely to have full-length. When 98% homology or more was observed for 80% region or more of cDNA clones to the registered clones, they were regarded as the same clone.

7) InterPro Homology Search

A protein domain search was performed by using information on the amino acid sequence of the rice full-length cDNA clones having the 28,332 deduced ORFs against the InterPro database. At the same time, similar InterPro searches were conducted using the following amino-acid sequence data to compare the both results.

Arabidopsis thaliana (27,288 sequences),

Caenorhabditis elegans (20,732),

Drosophila melanogaster (18,118),

Homo sapiens (24,147),

Saccharomyces cerevisiae (6360) , and

S. pombe (4962).

The search yielded a total of 3491 InterPro domains from all the above organisms. Table 4 highlights the 13 most frequent domains, and domains ranked below the third were observed almost commonly among 7 organism species. Second most frequently observed domains may contain the false hit data due to the small number of constitutive amino acid residues.

Among 3491 domains, 313 are plant (only O. sativa and A. thaliana)-specific and 1356 do not occur in plant proteins. There are 1177 domains that are animal (any of C. elegans, D. melanogaster, and H. sapiens) -specific, and 528 are not observed in these species. Eighty domains were yeast (S. cerevisiae and S. pombe) -specific and 1776 were not found in yeasts. These results indicate that 85% of the total number of domains is found in animals, 61% occur in plants, and 50% occur in yeasts. Comparison of the numbers of proteins having domains (number of clones) between rice and Arabidopsis yielded interesting results as described below.

Rice-specific domains (Table 4b), or those more frequent in rice than Arabidopsis (Table 4c),

Pollen allergic protein domain,

Organ-specifically expressed domains (e.g., serine protease inhibitor of seed),

Environmental stress-inducible proteins (antifreeze proteins, drought: ABA/WDS-inducible protein, etc.),

Domains more frequent specifically inArabidopsis (Table 4d)

Toll and interleukin-1 receptor (TIR) domain.

It was found that the TIR domain overwhelmingly occurs in the N-terminal region of the NBS-LRR type disease-resistance genes (R gene) products in Arabidopsis, but does not at all in rice. TIR-NBS-LRR type R genes are known to be amplified at high level in a specific genome region of Arabidopsis, but no alternative amplified domain was found in the R gene in rice. Transposon-related gene products were also more specifically and frequently found in Arabidopsis than in rice.

TABLE 4

| | | O. | | A. | | C. | | D. | | H. | | S. | | S. | |
|---|---|---|---|---|---|---|---|---|---|---|---|---|---|---|---|
| | | sativa | | thaliana | | elegans | | melanogaster | | sapience | | cereviciae | | pombe | |
| InterPro_ID | InterPro_name | count | rank | count | rank | count | rank | count | rank | count | rank | count | rank | count | rank |
| a. Top 13 | | | | | | | | | | | | | | | |
| IPR001687 | ATP/GTP- | 1281 | 1 | 1781 | 1 | 894 | 1 | 1061 | 1 | 1435 | 2 | 441 | 1 | 330 | 1 |
| | binding site | | | | | | | | | | | | | | |
| | motif A | | | | | | | | | | | | | | |
| | (P-loop) (*) | | | | | | | | | | | | | | |
| IPR000694 | Proline-rich | 1165 | 2 | 618 | 6 | 723 | 2 | 794 | 2 | 1791 | 1 | 88 | 6 | 51 | 10 |
| | region | | | | | | | | | | | | | | |
| IPR000719 | Eukaryotic | 944 | 3 | 1053 | 2 | 520 | 4 | 397 | 4 | 639 | 5 | 122 | 2 | 111 | 3 |
| | protein kinase | | | | | | | | | | | | | | |
| IPR002290 | Serine/Threonine | 827 | 4 | 1010 | 3 | 488 | 5 | 359 | 5 | 589 | 6 | 114 | 3 | 107 | 4 |
| | protein kinase | | | | | | | | | | | | | | |
| IPR001245 | Tyrosine protein | 792 | 5 | 995 | 4 | 402 | 8 | 340 | 6 | 557 | 8 | 106 | 4 | 101 | 5 |
| | kinase | | | | | | | | | | | | | | |
| IPR001611 | Leucine-rich | 344 | 6 | 504 | 7 | 68 | 51 | 151 | 16 | 219 | 23 | 6 | 46 | 10 | 37 |
| | repeat | | | | | | | | | | | | | | |
| IPR001841 | Zn-finger, RING | 341 | 7 | 436 | 9 | 155 | 22 | 145 | 19 | 320 | 13 | 34 | 19 | 44 | 11 |
| IPR001810 | Cyclin-like F-box | 335 | 8 | 650 | 5 | 428 | 6 | 46 | 63 | 70 | 69 | 14 | 38 | 13 | 34 |
| IPR000504 | RNA-binding | 325 | 9 | 313 | 10 | 160 | 20 | 293 | 7 | 340 | 10 | 65 | 11 | 86 | 6 |
| | region RNP-1 | | | | | | | | | | | | | | |
| | (RNA recognition | | | | | | | | | | | | | | |
| | motif) | | | | | | | | | | | | | | |
| IPR002885 | PPR repeat | 288 | 10 | 459 | 8 | 1 | 110 | 5 | 104 | 5 | 130 | 2 | 50 | 4 | 43 |
| IPR001680 | G-protein beta | 268 | 11 | 267 | 14 | 166 | 19 | 240 | 9 | 329 | 11 | 102 | 5 | 121 | 2 |
| | WD-40 repeat | | | | | | | | | | | | | | |
| IPR000379 | Esterase/lipase/ | 232 | 12 | 221 | 17 | 137 | 25 | 167 | 14 | 108 | 49 | 37 | 18 | 25 | 22 |
| | thioesterase, | | | | | | | | | | | | | | |
| | active site | | | | | | | | | | | | | | |
| IPR003593 | AAA ATPase | 227 | 13 | 307 | 11 | 116 | 27 | 160 | 15 | 159 | 31 | 82 | 7 | 70 | 7 |
| b. Exist | | | | | | | | | | | | | | | |
| in rice | | | | | | | | | | | | | | | |
| but not in | | | | | | | | | | | | | | | |
| Arabidopsis | | | | | | | | | | | | | | | |
| IPR005795 | Major pollen | 16 | 99 | | | | | | | | | | | | |
| | allergen Lol pI | | | | | | | | | | | | | | |
| IPR003496 | ABA/WDS | 15 | 100 | | | | | | | | | | | | |
| | induced protein | | | | | | | | | | | | | | |
| IPR000877 | Bowman-Birk | 13 | 102 | | | | | | | | | | | | |
| | serine protease | | | | | | | | | | | | | | |
| | inhibitor | | | | | | | | | | | | | | |
| c. Rice/ | | | | | | | | | | | | | | | |
| Arabi >> 1 | | | | | | | | | | | | | | | |
| IPR004873 | BURP domain | 70 | 51 | 5 | 125 | | | | | | | | | | |
| IPR001568 | Ribonuclease T2 | 29 | 86 | 5 | 125 | 1 | 110 | 1 | 108 | 1 | 134 | 1 | 51 | | |
| IPR000104 | Antifreeze | 69 | 52 | 13 | 117 | 4 | 107 | 143 | 20 | 41 | 94 | 5 | 47 | | |
| | protein, type I | | | | | | | | | | | | | | |
| d. Arabi/ | | | | | | | | | | | | | | | |
| Rice >> 1 | | | | | | | | | | | | | | | |
| IPR000157 | TTR domain | 1 | 114 | 126 | 29 | 3 | 108 | 12 | 97 | 23 | 112 | | | | |
| IPR004252 | Putative plant | 2 | 113 | 76 | 57 | | | | | | | | | | |
| | transposon | | | | | | | | | | | | | | |
| | protein | | | | | | | | | | | | | | |
| IPR004146 | DC1 domain | 4 | 111 | 138 | 25 | | | | | | | | | | |
| IPR003614 | Knottin | 1 | 114 | 19 | 111 | | | 6 | 103 | | | | | | |
| IPR000477 | RNA-directed | 5 | 110 | 91 | 47 | 64 | 54 | 33 | 76 | 69 | 70 | 5 | 47 | 13 | 34 |
| | DNA polymerase | | | | | | | | | | | | | | |
| | (Reverse | | | | | | | | | | | | | | |
| | transcriptase) | | | | | | | | | | | | | | |
| IPR005174 | Protein of | 2 | 113 | 34 | 96 | | | | | | | | | | |
| | unknown function | | | | | | | | | | | | | | |
| | DUF295 | | | | | | | | | | | | | | |
| IPR005162 | Retrotransposon | 7 | 108 | 86 | 50 | 5 | 106 | 2 | 107 | 1 | 134 | | | | |
| | gag protein | | | | | | | | | | | | | | |
| IPR003653 | SUMO/Sentrin/ | 8 | 107 | 81 | 52 | 6 | 105 | 7 | 102 | 7 | 128 | 2 | 50 | 4 | 43 |
| | Ubl1 specific | | | | | | | | | | | | | | |
| | protease | | | | | | | | | | | | | | |
| IPR001584 | Integrase, | 13 | 102 | 117 | 32 | 21 | 90 | 33 | 76 | 7 | 128 | 47 | 15 | 11 | 36 |
| | catalytic domain | | | | | | | | | | | | | | |
| IPR004332 | Plant MuDR | 13 | 102 | 99 | 42 | | | | | | | | | | |
| | transposase | | | | | | | | | | | | | | |

8) Transcription Factor

The InterPro search yielded 1336 transcription factor clones classified into 18 DNA-binding domains, as categorized in Table 5. As a result of classification, Zn finger-type transcription factors are most numerous, followed by Myb-type factors; these constitutions are similar to those for Arabidopsis. Of the predicted transcription factor clones, those included in the Zn finger-type are classified into subtypes as shown at the end of this specification.

TABLE 5

| | Number of | |
|---|---|---|
| Category | FL-cDNA clones | Comment |
| Zn finger | 588 | Including Ring, C2H2, Cx8Cx5C3H, |
| | | Dof, GATA, Constans, NF-X1 |
| Myb | 158 | |
| ERF | 83 | Including one ERF |
| NAM | 74 | |
| Homeo box | 73 | Including ELK, KNOX1,2 |
| bZIP | 63 | Not includind Zn finger |
| AUX/IAA | 47 | Including four TFB3 |
| WRKY | 46 | |
| TFB3 | 27 | Not including ERF, AUX/IAA |
| GRAS | 27 | Not including Zn finger |
| MADS | 26 | |
| HSF | 25 | |
| Tubby | 24 | |
| BRCT | 19 | Not including Zn finger |
| Fungal TF | 18 | |
| SBP | 17 | |
| Jumonji | 11 | Including jmjC, JmjN, not including |
| | | Zn finger |
| TCP(* | 10 | |
| Total | 1336 | |

9) Searches for Transmembrane Spanning Proteins and Their Cellular Location

The MEMSAT programis effective in predicting the secondary structure of a transmembrane spanning proteins and their topology. The use of this program identifies the number of transmembrane spanning segments of full-length CDNA clones and their topology. Table 6 shows the number of transmembrane-spanning segments, total number of said clones, and direction of the segments (when the N-terminus is intracellular: IN, when extracellular: OUT).

TABLE 6

| | | |
|---|---|---|
| Number of | | Spanning |
| transmembrane-spanning | Total number | direction |

| segment | of clone | In | Out |
|---|---|---|---|
| 1 | 17839 | 8175 | 9664 |
| 2 | 2842 | 1585 | 1257 |
| 3 | 1259 | 646 | 613 |
| 4 | 789 | 470 | 319 |
| 5 | 383 | 160 | 223 |
| 6 | 290 | 177 | 113 |
| 7 | 256 | 90 | 166 |
| 8 | 109 | 62 | 47 |
| 10 | 138 | 97 | 41 |
| 11 | 120 | 59 | 61 |
| 13 | 51 | 14 | 37 |
| 14 | 19 | 18 | 1 |
| 15 | 4 | 3 | 1 |
| 16 | 12 | 7 | 5 |
| 17 | 5 | 3 | 2 |
| 20 | 3 | 3 | 0 |

It was found that 17,839 clones had only one transmembrane-spanning segment, while 6,280 clones had two or more, accounting for 22.1% of the total cDNA clones. As to the spanning direction, one direction overwhelmingly exceeds the other in number depending on the number of spannings in some cases, but the numbers of both directions were mostly about the same.

The pSORT program is effective in predicting signal peptide sequences and their cellular location. This program yields the data on the putative target organelles to be sorted and the certainity of the localization. The certainity ranged from 0.9968 to 0.2 in the trial analysis. When, a cut-off value for the certainity was set at 0.5, this sorting process predicted the localization of signal peptide sequences in 18,166 clones out of those with ORF having 100 or more amino acid residues. Table 7 shows the predicted target organelles, number of clones whose localization in that target has been predicted, and the ratio (%) of said clones to 18,166 clones. Proteins predicted to be in the nucleus formed the largest group, accounting for 20.0% of total, followed by those in the plasma membrane, cytoplasm, endoplasmic reticulum, microbody, etc., and these locations all accounted for about 10%.

TABLE 7

| Target organella | | Number of clones | (%) |
|---|---|---|---|
| Chloroplast | Stroma | 1383 | 7.6 |
| | thylakoid membrane | 542 | 3.0 |
| | thylakoid space | 90 | 0.5 |
| Cytoplasm | | 1875 | 10.3 |
| ER | | 2045 | 11.3 |
| Golgi body | | 26 | 0.1 |
| Microbody | | 1783 | 9.8 |
| Mitochondorion | inner membrane | 502 | 2.8 |
| | Intermembrane space | 80 | 0.4 |
| | matrix space | 1434 | 7.9 |
| | outer membrane | 32 | 0.2 |
| Nuleus | | 3635 | 20.0 |
| Outside | | 1508 | 8.3 |
| Plasma membrane | | 2982 | 16.4 |
| Vacuole | | 249 | 1.4 |
| Total | | 18166 | |

A cut-off value for certainity: >0.5

10) Homology With Arabidopsis

To examine the homology of the full-length cDNA clones to the genes encoded in the Arabidopsis genome, rice 28,444 ORF amino-acid sequences were compared with the 27,288 deduced amino-acid sequences from the predicted CDS of the Arabidopsis genome at the BLASTP threshold of E<10⁻⁷. Results are summarized in Table 8. 18,900 full-length CDNA clones (12,996 TU, 64%) had a homologin Arabidopsis, and 20,473 Arabidopsis genes (75%) had a homolog in the rice full-length cDNAs.

Similar comparison of the indica genome with Arabidopsis genes at the genome sequence level was already reported, saying that 50% of predicted rice genes had a homolog in Arabidopsis and 80% of Arabidopsis genes had a homolog in rice. If the prediction in indica is correct, then considering the full-length cDNA clones being 19,000 TU and the redundancy of genes in their respective genomes, it becomes a subject to further acquire a 5% equivalent gene family common to Arabidopsis and collect more rice-specific gene family to reduce the fraction of Arabidopsis-common TU from 64% to 50%.

TABLE 8

| | | japonica | | | indica | | | Arabidopsis | | |
|---|---|---|---|---|---|---|---|---|---|---|
| Fraction | | E<-10 | E<-50 | E<-100 | E<-10 | E<-50 | E<-100 | E<-10 | E<-50 | E<-100 |
| 1 | japonica | 2667 | 6698 | 12073 | *** | *** | *** | *** | *** | *** |
| | specific | | | | | | | | | |
| 2 | indica | *** | *** | *** | 18461 | 30655 | 38684 | *** | *** | *** |
| | specific | | | | | | | | | |
| 3 | Arabidopsis | *** | *** | *** | *** | *** | *** | 5171 | 12148 | 18129 |
| | specific | | | | | | | | | |
| 4 | japonica & | 5560 | 7627 | 8682 | 8215 | 7969 | 7252 | *** | *** | *** |
| | indica | | | | | | | | | |
| | common | | | | | | | | | |
| 5 | indica & | *** | *** | *** | 1553 | 1094 | 777 | 1644 | 1667 | 1445 |
| | Arabidopsis | | | | | | | | | |
| | common | | | | | | | | | |
| 6 | Arabidopsis | 372 | 610 | 622 | *** | *** | *** | 674 | 983 | 1036 |
| | & japonica | | | | | | | | | |
| | common | | | | | | | | | |
| 7 | common | 20351 | 14015 | 7573 | 25169 | 13680 | 6685 | 19799 | 12490 | 6678 |
| | amang | | | | | | | | | |
| | three | | | | | | | | | |
| | total | | 28950 | | | 53398 | | | 27288 | |

11) Functional Classification of Genes Using Gene Ontology (GO)

GO terms are assigned to GenBank reports, InterPro domains and Arabidopsis genes. If GO terms are assigned to the information in the concerned database to which full-length cDNAs of the present invention showed homology, GO terms can be added to the full-length cDNA clones of the present invention based on the results of homology search. Therefore, these GO terms were collected to perform the functional classification based on them.

The total number of cDNA clones of the present invention to which GO terms associated with “biological processes” were added was 18,485, which were classified as shown in Tables 9-12. About a half of full-length cDNAs with “unclassified function” formed the largest group, followed by those classified into “metabolism,” “transport,” “translation,” , accounting for one-fourth of the total terms. GO terms associated with “function” were added to 10,942 full-length cDNA clones, and the total number of said GO terms was 16,853. These GO terms are unable to be classified exclusively, and the most frequently observed GO “function” term was “enzyme.” GO terms associated with “cellular component” were added to 3629 full-length cDNA clones, and the total number of said GO terms was 3637

TABLE 9

| Category | Number | % |
|---|---|---|
| unclassified | 9869 | 53.4 |
| Metabolism | 4912 | 26.6 |
| Transport | 1108 | 6.0 |
| Translation | 694 | 3.8 |
| Transcription | 583 | 3.2 |
| cell communication | 436 | 2.4 |
| Energy | 276 | 1.5 |
| Communication, Defense | 241 | 1.3 |
| Cell growth Maintenance | 164 | 0.9 |
| Developmental Process, Aging, Death | 101 | 0.5 |
| DNA replication | 92 | 0.5 |
| others | 9 | 0.0 |
| total | 18485 | |

TABLE 10

| Category | Number |
|---|---|
| enzyme | 5988 |
| ligand binding or carrier | 5369 |
| nucleic acid binding | 2199 |
| transporter | 1615 |
| structural protein | 499 |
| molecular_function unknown | 276 |
| signal transducer | 193 |
| enzyme inhibitor | 143 |
| defense/immunity protein | 112 |
| motor | 60 |
| chaperone | 48 |
| storage protein | 41 |
| toxin | 12 |
| microtubule binding | 8 |
| enzyme activator | 8 |
| apoptosis regulator | 5 |
| obsolete | 4 |
| cell adhesion molecule | 3 |
| Total | 16583 |

TABLE 11

| Enzymes | Number |
|---|---|
| hydrolase | 2099 |
| transferase | 1909 |
| kinase | 1126 |
| oxidoreductase | 796 |
| ATPase | 378 |
| lyase | 250 |
| phosphatase | 213 |
| ligase | 185 |
| helicase | 130 |
| monooxygenase | 105 |
| isomerase | 87 |
| small protein conjugating enzyme | 50 |
| aldolase | 37 |
| serine esterase | 35 |
| disulfide oxidoreductase | 25 |
| small protein activating enzyme | 7 |
| glycine cleavage system | 6 |
| heme-copper terminal oxidase | 6 |
| Rieske iron-sulfur protein | 4 |
| 1-deoxyxylulose-5-phosphate synthase | 3 |
| 3,4 dihydroxy-2-butanone-4-phosphate synthase | 3 |
| lipoate-protein ligase | 3 |
| 2C-methyl-D-erythritol 2,4-cyclodiphosphate synthase | 1 |
| DNA repair enzyme | 1 |
| glycogen debranching enzyme | 1 |
| imidazoleglycerol-phosphate synthase | 1 |
| Total | 7461 |

TABLE 12

| Location | Number |
|---|---|
| cell | 3414 |
| unlocalized | 165 |
| extracellular | 56 |
| external protective structure | 2 |
| cellular_component unknown | 0 |
| obsolete | 0 |
| Total | 3637 |
| intracellular | 2222 |
| membrane | 1457 |
| Total (cell) | 3679 |

12) Tissue-Specifically Expressed Gene

Full-length CDNA clones of the present invention were obtained from various libraries as shown in Table 1. Comparing genes obtained among these libraries with each other enables to sort genes universally obtained from a plurality of tissues and those specifically expressed in particular tissues. The results obtained by this gene expression analysis are referred to as body map.

The body map of the full-length cDNAs of the present invention revealed that most of full-length cDNA clones obtained from libraries derived from the flower organs are specifically xpressed in the flower organs and are not expressed in other tissues. A list of clones derived from flower organs is shown as “Clone list derived from flower organs” at the end of this specification. Most of clones set forth in this list are genes whose expressions have been confirmed in the maturation processes from the stages of young spikes through seed maturation.

13) Conclusion

The rice full-length cDNA project has completely sequenced a set of 28,469 clones and mapped them to the genome, revealing that these clones are those derived from at least 19,000 TU. Information on these cDNA clones will provide precise gene annotation from the genome sequence data, and facilitate the assignment of EST sequences to the corresponding promoter sequences. Considering possible bias during collection of various clones, the frequency of alternative splicing events in plants may be lower than that in animals.

Many plant genes are known to be amplified compared to those in animals, suggesting differences between plants and animals in the strategies used to increase the variety of proteins. Protein informatics analyses using the InterPro database provide insight into differences in the profile of the plant proteins such as proteins more frequent in rice than Arabidopsis and vice versa.

The protein homology searches between rice and Arabidopsis confirmed that the present full-length cDNA collection contains homologs corresponding to 75% of genes predicted in Arabidopsis, while 64% of the TU clusters in rice are homologous to Arabidopsis gene.

“Clone List”

The Clone list (Table 13; submitted in electronic format) shows, from the left column, the clone name (CLONE_NAME), DDBJ accession number (DDBJ_ACC), nucleotide sequence identification number (N_ID), amino acid sequence identification number (AMINO_ID), transcription initiation codon position (START_POSITION) and transcription termination codon position (STOP_POSITION).

“BLAST X Search Results”

The following information on each clone was described in Table 14 (submitted in electronic format) from the left divided by //.

Clone name,

DDBJ accession number of each clone,

Cluster ID to which the clone belongs,

Accession number of clone which yielded hits in BLAST N,

Definition of clone that yielded hits in BLAST N,

Score when yielding hits in BLAST N,

E value when yielding hits in BLAST N,

Accession number of clone that yielded hits in BLAST X,

Definition of clone that yielded hits in BLAST X,

Score when yielding hits in BLAST X,

E value when yielding hits in BLAST X

“List of Clones Predicted as Transcription Factor”

In the List of Clones Predicted as Transcription Factors (Table 15; submitted in electronic format), Clone name, ID of InterPro domain name, and domain name are described from the left divided by =.

“List of Clones Predicted to be Zn Finger”

Results of categorizing clones predicted to be Zn finger into subtypes are shown. Corresponding clones are listed following the InterPro domain ID and domain name.

[IPR000822;Zn-Finger, C2H2 Type]

001-012-G08, 001-014-E06, 001-015-H09, 001-017-D01, 001-017-F12, 001-017-G09, 001-019-B08, 001-021-A06, 001-021-F09, 001-023-A03, 001-030-G02, 001-038-B11, 001-102-C06, 001-103-H07, 001-107-E09, 001-111-B01, 001-111-H03, 001-113-E02, 001-113-E12, 001-119-H03, 001-125-A08, 001-128-E09, 001-200-D09, 001-200-H06, 001-203-E10, 002-102-A12, 002-107-D06, 002-107-G07, 002-116-B04, 002-116-F05, 002-119-B08, 002-119-G10, 002-129-B01, 002-130-B07, 002-131-D01, 002-137-A12, 002-139-A09, 002-139-C06, 002-140-F08, 002-141-H01, 002-144-D12, 002-148-B03, 002-148-B06, 002-150-D08, 002-151-G02, 002-153-F01, 002-153-G07, 002-155-A06, 002-159-D08, 002-160-H02, 002-160-H09, 002-161-H07, 002-162-A11, 002-162-A12, 002-167-B07, 002-173-CO5, 002-173-H08, 002-181-F05, 006-206-D03, 006-207-E08, 006-212-C04, 006-311-A06, J013000K02, J013001C12, J013001G09, J013002J08, J013026K11, J013031H23, J013047G22, J013050J04, J013056E07, J013056L24, J013073B17, J013089B09, J013095D10, J013097L08, J013104D22, J013105K20, J013110K19, J013124C13, J013126A02, J013127D16, J013130L03, J013131B08, J013149G12, J013161E15, J013161F12, J013165P15, J013170H07, J023001N18, J023004M17, J023006J01, J023007J12, J023014K13, J023022G20, J023023L10, J023023M18, J023026P04, J023050E05, J023055E06, J023055J24, J023078D20, J023079017, J023085P15, J023088A12, J023093M20, J023109C14, J023109M13, J023110G13, J023114G08, J023119J10, J023123F03, J023123L01, J023125M06, J023142J09, J033029F02, J033045B09, J033046D05, J033054017, J033074H23, J033075E02, J033081C08, J033084A01, J033085L23, J033086G06, J033098G15, J033101B01, J033101Hl9, J033115G09, J033121H10, J033121H12, J033129E14, J033143G04, J033147N02

[IPR002926;Zn-Finger, CONSTANS Type]

001-007-G06, 001-029-D01, 001-205-D08, 002-118-C11, 006-205-E01, 006-303-A11, J013001A08, J013117D12, J013152B04, J023001E21, J023090D24, J023105D03

[IPR000571;Zn-Finger, C-x8-C-x5-C-x3-H type] 001-014-C04, 001-020-C06, 001-027-BlO, 001-030-A08, 001-032-B06, 001-039-A01, 001-044-E06, 001-047-F12, 001-202-E04, 001-204-A04, 001-205-D09, 001-206-H10, 002-102-A03, 002-102-F01, 002-103-E06, 002-107-G01, 002-110-B05, 002-111-A03, 002-120-E10, 002-131-G11, 002-140-H10, 002-141-G08, 002-155-C09, 002-159-BO5, 002-163-E09, 002-164-G05, 002-169-F02, 002-179-E04, 002-182-H10, J013000H23, J013001J05, J013002B05, J013002E19, J013025G09, J013050D10, J013056L03, J013094C18, J013114A13, J013116A14, J013116H24, J013123G12, J013159G03, J023003014, J023009A16, J023038J07, J023039E23, J023041B11, J023066C11, J023078K23, J023082B02, J023090K17, J023091E18, J023092H03, J023093012, J023095J08, J023119N23, J033043A02, J033045L14, J033050C24, J033073E09, J033090J15, J033099F01, J033102J01, J033114B10, J 033145I17

[IPR003851;Zn-Finger, Dof Type]

001-028-A11, 001-032-E07, 001-035-D05, 001-113-G11, 001-114-F11, 002-126-C02, 002-129-F12, 002-153-A09, 002-155-A04, 006-203-F09, 006-303-F03, J013026L11, J013041J16, J013091E10, J013152P10, J013155H18, J023060H13, J023076F14, J033034E07

(IPR000679;Zn-Finger, GATA Type]

001-011-G08, 001-023-D04, 001-200-C09, 002-162-F07, J013048G01, J013064I12, J013120L22, J013136H07, J023003E02, J023034D16, J023055K24, J023063M07, J033033F09, J033038C04, J033044C20, J033058P14, J033068D01

[IPR000967;Zn-Finger, NF-X1 type]

002-161-C10, 002-162-G03, 002-166-F06, J013060014, J013082G02

[IPR001841;Zn-Finger, RING]

001-001-C07, 001-001-F04, 001-002-E12, 001-003-C12, 001-005-B04, 001-006-B04, 001-007-D07, 001-010-F10, 001-012-E09, 001-014-D03, 001-017-F04, 001-018-E04, 001-019-H07, 001-022-D10, 001-023-B01, 001-023-C12, 001-025-F02, 001-025-H03, 001-025-H09, 001-027-B09, 001-028-B04, 001-031-A11, 001-032-H11, 001-033-A01, 001-033-B02, 001-035-B09, 001-035-H12, 001-036-A02, 001-037-B09, 001-039-B04, 001-039-G03, 001-040-F07, 001-040-G01, 001-043-F05, 001-045-D12, 001-045-H12, 001-105-D02, 001-107-A12, 001-110-A08, 001-112-G10, 001-112-H07, 001-113-G05, 001-114-F01, 001-115-G04, 001-116-C07, 001-118-F06, 001-121-E06, 001-122-C02, 001-123-D08, 001-124-C08, 001-127-C08, 001-200-C08, 001-204-D04, 001-204-D05, 001-205-B01, 001-205-F11, 001-206-A03, 001-206-B05, 001-206-E07, 001-208-D10, 002-101-A07, 002-101-G06, 002-104-G10, 002-107-C03, 002-107-D06, 002-107-D08, 002-107-G12, 002-108-A09, 002-108-E10, 002-108-G02, 002-108-H08, 002-108-H11, 002-110-F12, 002-113-C04, 002-116-G03, 002-118-C12, 002-118-G07, 002-124-E05, 002-124-E07, 002-131-F11, 002-132-E06, 002-135-A09, 002-137-A02, 002-137-C02, 002-138-B01, 002-139-E12, 002-140-H11, 002-141-F12, 002-142-C10, 002-143-E10, 002-143-G02, 002-147-B06, 002-149-F01, 002-150-E02, 002-152-A08, 002-152-A11, 002-152-H11, 002-153-B07, 002-154-D02, 002-154-F07, 002-155-B05, 002-155-G01, 002-162-B04, 002-162-H03, 002-164-B11, 002-164-D12, 002-166-D03, 002-166-F06, 002-167-G10, 002-173-B09, 002-173-B10, 002-174-H07, 002-176-C01, 002-176-D08, 002-177-E02, 002-178-C01; 002-188-G12, 006-201-H12, 006-202-G09, 006-203-C10, 006-204-C11, 006-204-E05, 006-205-F10, 006-207-G05, 006-209-G12, 006-210-E12, 006-211-B01, 006-212-C03, 006-212-D11, 006-212-F09, 006-212-H10, 006-301-D04, 006-301-G04, 006-302-G11, 006-305-B01, 006-307-D05, 006-308-A03, 006-308-C09, 006-308-C10, 006-310-A03, 006-311-G08, J013000B10, J013000P06, J013001J11, J013001N16, J013002F03, J013002G21, J013002N04, J013002N07, J013002P08, J013014C10, J013020F11, J013024P14, J013027C16, J013028F14, J013030E05, J013030E23, J013033B12, J013033K23, J013039J02, J013041Kl9, J013050C14, J013052012, J013052P13, J013057F24, J013058G19, J013058N02, J0130591 17, J013059J01, J013061F22, J013063D05, J013064M13, J013065E10, J013069F21, J013073A02, J013074C24, J013082G02, J013082L02, J01308415, J013089D16, J013090G16, J013091H18, J013093E22, J013093J16, J013094L24, J013095B01, J013095L18, J013096G12, J013096J16, J013097A17, J013097C11, J013103K22, J013104C20, J013104I23, J013104014, J013107I10, J013111A10, J013111A16, J013112G09, J013113E15, J013115015, J013115P09, J013116F16, J013116G13, J013116I02, J013116L22, J013119B08, J013121E16, J013128Jl9, J013130B11, J013130C12, J013130E06, J013131L14, J013134A03, J013135G08, J013144A04, J013145E21, J013153F20, J013157D07, J013157F12, J013159H10, J013160D07, J013169C17, J013169J03, J023001G18, J023003A15, J023005I07, J023009D02, J023009007, J023010E11, J023010H14, J023011H14, J023018C07, J023019021, J023020D18, J023020P04, J023021A17, J023023M16, J023031B11, J023031G24, J023034D08, J023039004, J023041E24, J023044E12, J023044P07, J023047F13, J023047F14, J023049F08, J023049J20, J023052D05, J023052J15, J023054E15, J023054P08, J023061K06, J02306318, J023066I04, J023072K15, J023075N19, J023077E12, J023077P18, J023078L19, J023089D17, J023096P15, J023097G23, J023098C19, J023105K15, J023105K22, J023106F04, J023106J06, J023106M02, J023107E20, J023108I11, J023110J02, J023114C23, J023124E09, J023127C10, J023133D04, J023134P18, J023139M11, J023142C17, J023148H24, J023149D24, J023149P20, J023150J15, J033000G10, J033010L07, J033015K15, J033020F05, J033020J08, J033021A14, J033023F07, J033023K06, J033025K23, J033026G05, J033026H11, J033029A20, J033030D14, J033033J06, J033036L15, J033038J15, J033041L15, J033047I01, J033048I07, J033048J15, J033050H01, J033051D17, J033058G15, J033060L17, J033063D14, J033067G01, J033068I24, J033068K11, J033068N07, J033069D23, J033072K04, J033073H23, J033075P09, J033083D05, J033084A01, J033088P15, J033089H07, J033090H12, J033091I10, J033094D12, J033094G14, J033101B01, J033104F07, J033106E23, J033107017, J033108E06, J033117A10, J033119F23, J033119L05, J033120I17, J033122C24, J033125G22, J033129G15, J033132P09, J033133Fl9, J033142Nl6, J033149F0l

“Results of Gene Ontology”

Clone name (CLONE_NAME), database name, GO code, and gene ontology label (GO_LABEL) were described in Table 16 (submitted in electronic format) according to GO categories divided by =.

“List of Clones Included in Each Category of Gene Ontology”

Clone names and GO terms added to respective clones are categorized.

Binding Protein[Binding]

J023051M04==actin binding
J023149A14==acyl-CoA binding
J023030G12==amino acid binding
002-144-D12==ATP binding
002-145-D12==ATP-binding cassette (ABC) transporter
J013002C11==biotin binding
002-143-H11==calcium ion binding
002-154-C06==chitin binding
002-160-B03==chromatin binding
002-149-C02==copper binding
002-143-H02==DNA binding
J023111A18==double-stranded RNA binding
J023029A13==GTP binding
J023033I19==heavy metal binding
J023079P11==iron binding
J023041I08==lipid binding
002-150-D04==microtubule binding
002-143-F09==nucleic acid binding
002-147-D09==nucleotide binding
002-145-H10==protein binding
002-145-G05==RNA binding
002-143-E10==sugar binding
002-137-H02==tRNA binding
002-143-E06==zinc binding
   (Enzyme_Inhibitor]
J023043J23==cysteine protease inhibitor
J013043C13==endopeptidase inhibitor
002-151-F03==enzyme inhibitor
J023100009==RAB GDP-dissociation inhibitor
J023112G04==Rho GDP-dissociation inhibitor
002-145-A10==serine protease inhibitor
   Transporter Protein [Transporter]
J023037N07==amino acid-polyamine transporter
002-150-A12==ammonium transporter
002-145-D12==ATP-binding cassette (ABC) transporter
J023036P06==cation transporter
J013108A03==cobalt ion transporter
002-144-C02==electron transporter
J023054A11==heavy metal ion transporter
002-111-D08==heme transporter
J023028L13==hydrogen-transporting two-sector ATPase
J023050L03==inorganic phosphate transporter
J023136I24==intracellular transporter
002-169-D04==nucleobase transporter
J033024P16==nucleoside transporter
J023052011==nucleotide-sugar transporter
002-151-F01==plasma membrane cation-transporting ATPase
J023055A03==potassium transporter
J023048B11==protein transporter
   [Carrier]
J023122I22==acyl carrier
J033133F17==carrier
002-150-B07==Fe3S4/Fe4S4 electron transfer carrier
J023035H08==iron-sulfur electron transfer carrier
J023034G24==protein carrier
J023028D17==redox-active disulfide bond electron carrier
   [Enzyme]
00l-100-C02==“1,3-beta-glucan synthase”
J023080J16==1-aminocyclopropane-1-carboxylate synthase
J023132G24==1-deoxyxylulose-5-phosphate synthase
006-308-E05==“1-phosphatidylinositol-4,5-bisphosphate phosphodiesterase”
J023083N15==1-phosphatidylinositol-4-phosphate 5-kinase
001-003-D03==“2C-methyl-D-erythritol 2,4-cyclodiphosphate synthase”
002-145-A06==2-dehydro-3-deoxyphosphoheptonate aldolase
J023144M13==“3,4 dihydroxy-2-butanone-4-phosphate synthase”
J023083L07==3′-5′ exoribonuclease
J013032A17==3-beta-hydroxy-delta(5)-steroid dehydrogenase
J033028020==3-dehydroquinate dehydratase
J013067A20==3-dehydroquinate synthase
J013000009==3-deoxy-manno-octulosonate cytidylyltransferase
J033004006==3-hydroxyisobutyrate dehydrogenase
002-151-H05==5-methyltetrahydropteroyltriglutamate-homocys teine S-methyltransferase
J023050A11==6-phosphofructokinase
002-138-C02==acetolactate synthase
002-154-A08==acetyl-CoA carboxylase
J023043F24==acetylglutamate kinase
J023087C13==acid phosphatase
002-154-G06==acyl-CoA dehydrogenase
006-301-D08==acyl-CoA oxidase
006-307-C01==acyl-CoA thioesterase
002-147-G01==acyltransferase
006-203-H06==adenosine kinase
J013070D05==adenosinetriphosphatase
J013002P17==adenosylhomocysteinase
J023041P13==adenosylmethionine decarboxylase
J013088J03==adenylosuccinate synthase
002-177-B04==alanine-tRNA ligase
002-144-E03==“alcohol dehydrogenase, zinc-dependent”
J023139A20==aldolase
J023050C09==aldose 1-epimerase
002-139-H02==“alpha,alpha-trehalase”
002-145-G09==“alpha,alpha-trehalose-phosphate synthase (UDP-forming)”
J033046P17==“alpha-1,3-mannosylglycoprotein beta-1,2-N-acetylglucosaminyltransferase”
002-162-A01==alpha-amylase
002-167-G08==alpha-mannosidase
J023039El2==amidase
006-204-B10==aminoacyl-tRNA hydrolase
J013106I02==aminomethyltransferase
006-208-D05==aminopeptidase
J023082J11==ammonia ligase
J013052F22==arginine-tRNA ligase
J023081B09==argininosuccinate synthase
006-309-E04==asparaginase
J033004I10==aspartate kinase
002-160-A04==aspartate-tRNA ligase
002-149-B12==aspartic-type endopeptidase
J033082C14==ATP phosphoribosyltransferase
002-161-E09==ATP-dependent peptidase
J023048N01==beta-amylase
J033105C09==beta-galactosidase
J023055K01==beta-N-acetylhexosaminidase
002-159-A07==biotin carboxylase
J033031K02==biotin synthase
J013167021==calpain
J033082C21==carbamoyl-phosphate synthase
002-152-A12==carbonate dehydratase
002-161-G04==carboxyl- and carbamoyltransferase
J023068K15==carboxy-lyase
002-147-C06==carboxypeptidase A
J013051E04==“casein kinase II, regulator”
002-174-H07==caspase
J023079C18==catalase
J023041M10==CDP-diacylglycerol-glycerol-3-phosphate
3-phosphatidyltransferase
002-164-A06==chitin synthase
J023042L15==chi tinase
J033031C20==chorismate mutase
J013110C10==chorismate synthase
J023146L07==citrate (SI)-synthase
J023088103==CoA hydrolase
002-162-H04==“copper, zinc superoxide dismutase”
J023050L0 8==coproporphyrinogen oxidase
001-102-A03==cyanate lyase
J013071P10==cysteine-tRNA ligase
002-143-E04==cysteine-type endopeptidase
J023089B16==cytochrome c oxidase
J023148P17==D-alanine-D-alanine ligase
J033051A22−=deoxyhypusine synthase
002-182-G05==deoxyribodipyrimidine photolyase
J023080K21==diacylglycerol kinase
001-046-A07==dihydrodipicolinate reductase
J023089118==dihydroneopterin aldolase
J013002H08==dihydroorotate dehydrogenase
J013147H17==dihydropteroate synthase
J023030K24==disulfide oxidoreductase
006-203-E05-=DNA 3-methyladenine glycosylase I
002-160-D05==DNA ligase (ATP)
033.020H02==DNA photolyase
002-151-C09=-DNA-directed RNA polymerase
J033003F07==dolichyl-diphospho-oligosaccharide-protein glycosyltransferase
J033038E19==endonuclease
J023031C24==endopeptidase C1p
J023054N24==exonuclease
001-102-D09==ferredoxin reductase
J033072J07==ferrochelatase
J023125G01==folylpolyglutamate synthase
002-164-E10==formate-tetrahydrofolate ligase
002-119-H07=-formylmethionine deformylase
J023038B06==fructose-bisphosphate aldolase
J013071F22==fucosyltransferase
J023071K13==galactosylgalactosylxylosylprotein 3-beta-glucuronosyltransferase
J023065E21==galactosyltransferase
J033088N13==glucose-1-phosphate adenylyltransferase
J023102M14==glucose-6-phosphate 1-dehydrogenase
J033116J21==glucose-6-phosphate isomerase
J033069K09-=glutamate N-acetyltransferase
002-166-H10==glutamate synthase
J033031H21==glutamate-5-semialdehyde dehydrogenase
006-208-G06==glutamate-ammonia ligase
J023141A17==glutamate-tRNA ligase
J023058E10==glutamine amidotransferase
001-047-D06==-glutamyl tRNA reductase
J013000L07==glutamyl-tRNA(Gln) amidotransferase
J033076L03==glutathione peroxidase
J013033N23==glutathione synthase
J023036H06==glyceraldehyde 3-phosphate dehydrogenase (phosphorylating)
002-159-C07==glycerol-3-phosphate dehydrogenase
J023050E11==glycerol-3-phosphate dehydrogenase (NAD+)
J013124A06==glycerone kinase
J023047B14==glycerophosphodiester phosphodiesterase
002-161-H05==glycine dehydrogenase (decarboxylating)
002-153-G10==glycine hydroxymethyltransferase
J023135F01==glycine-tRNA ligase
002-155-F06==glycolipid 2-alpha-mannosyltransferase
J023044M15==glycosyltransferase
J033040C05==glycyl-peptide N-tetradecanoyltransferase
001-201-F05==GTP cyclohydrolase I
J013116D09==GTP cyclohydrolase II
002-167-C06==GTPase
002-165-D05==guanylate cyclase
002-179-F05==heterotrimeric G-protein GTPase
J023031J07==“heterotrimeric G-protein GTPase, alpha-subunit”
J023038N14==hexokinase
J023037L20==histidinol dehydrogenase
J033030E05==homocysteine S-methyltransferase
J023098E10==homoserine dehydrogenase
001-020-B10==homoserine kinase
J023047F16==-hydrogen-translocating pyrophosphatase
J023086K07==hydrogen-translocating V-type ATPase
J023028L13==hydrogen-transporting two-sector ATPase
J023031L04==hydrolase
J023030L13==“hydrolase, acting on carbon-nitrogen (but not peptide) bonds”
J023114011==“hydrolase, acting on carbon-nitrogen (but not peptide) bonds, in cyclic amides”
J023133M21==“hydrolase, acting on carbon-nitrogen (but not peptide) bonds, in cyclic amidines”
J013059B20==“hydrolase, acting on carbon-nitrogen (but not peptide) bonds, in linear amides”
002-145-B07==“hydrolase, hydrolyzing O-glycosyl compounds”
002-159-G02==hydro-lyase
J023089B21==“hydroxymethyl-, formyl- and related transferase”
J033088M20==hydroxymethylbilane synthase
J013170018==hydroxymethylglutaryl-CoA lyase
J033025C16==hydroxymethylglutaryl-CoA reductase (NADPH)
J023079L19==hydroxymethylglutaryl-CoA synthase
J033025I19==imidazolegLycerol-phosphate dehydratase
J033107D20==IMP cyclohydrolase
J023092E01==indole-3-glycerol-phosphate synthase
J023047P17==inositol/phosphatidylinositol kinase
002-148-A06==inositol/phosphatidylinositol phosphatase
J023l25K01==inositol-3-phosphate synthase
J023034C14==“intramolecular transferase, phosphotransferases”
002-168-C03==isopentenyl-diphosphate delta-isomerase
J013002N13==ketol-acid reductoisomerase
J033057L23==kinase
J023043J03==lactoylglutathione lyase
002-160-B02==leucine-tRNA ligase
J023132C04==lipid-A-disaccharide synthase
J033066K16==lipoate synthase
J023043P03==lyase
002-165-A01==lysine-tRNA ligase
002-164-F03==lysozyme
J023056M13==magnesium chelatase
002-172-H02==malate dehydrogenase
J023065M02==malic enzyme
J023043B18==mannose-6-phosphate isomerase
002-166-F07==“mannosyl-oligosaccharide 1,2-alpha-mannosidase”
002-159-E10==mannosyltransferase
J023039G20=metalloendopeptidase
001-040-H12==metalloexopeptidase
J023028L23==metallopeptidase
J023095C13==methionine adenosyltransferase
J023031N01==methyltransferase
002-144-B01==monooxygenase
J023100D05==N-acetyl-gamma-glutamyl-phosphate reductase
002-166-B07==N-acetyltranferase
001-116-B03==NAD+ ADP-ribosyltransferase
001-113-E04==NAD+ synthase (glutamine-hydrolyzing)
J023048E09==NADH dehydrogenase (ubiquinone)
J023055E24==nicotianamine synthase
J023049111==nuclease
J023039G14==nucleoside-diphosphate kinase
J023056M07==nucleotidyltransferase
J023054H04==oligosaccharyl transferase
006-203-H05==O-methyltransferase
J033094F19==orotidine-5′-phosphate decarboxylase
J023064I01==O-sialoglycoprotein endopeptidase
002-147-D07==oxidoreductase
002-170-H05==“oxidoreductase, acting on the aldehyde or oxo group of donors, disulfide as acceptor”
001-040-G05==pantoate-beta-alanine ligase
002-146-G03==pepsin A
J023038A03==peptidase
J023033M17==peroxidase
002-155-B07==phenylalanine-tRNA ligase
J023114K10==phosphatidate cytidylyltransferase
J023108I08==phosphatidylcholine-sterol O-acyltransferase
J013008I04==phosphatidylserine decarboxylase
002-147-B03==phosphoenolpyruvate carboxykinase (ATP)
J023049F03==phosphoenolpyruvate carboxylase
J023080N03==phosphogluconatedehydrogenase (decarboxylating)
J023036N24==phosphoglycerate kinase
002-131-H11==phospholipase
001-039-E07==phospholipase A2
J023050004==phospholipase C
006-304-H09==phosphomannomutase
J023133I04==phosphopyruvate hydratase
J023114015==phosphoribosylamine-glycine ligase
J013129D15==phosphoribosylaminoimidazole carboxylase
001-115-F04==phosphoribosylaminoimidazole-succinocarboxamide synthase
006-203-G09==phosphoribosyl-AMP cyclohydrolase
001-121-B07==phosphorylase
002-174-E12==phosphoserine phosphatase
002-151-F01==plasma membrane cation-transporting ATPase
002-143-E12==polygalacturonase
J033069C18==porphobilinogen synthase
002-162-D12==prephenate dehydratase
J013159D01==prephenate dehydrogenase (NADP+)
J013002B07==procollagen-lysine 5-dioxygenase
J023012J21==prolyl oligopeptidase
J023041C17==proteasome endopeptidase
002-145-E02==protein kinase
J023079D17==protein phosphatase
J023060H13==protein prenyltransferase
001-044-A03==protein serine/threonine kinase
J023031I12==protein serine/threonine phosphatase
002-143-E05==protein translocase
002-155-E07==protein tyrosine kinase
002-162-F06==protein tyrosine phosphatase
002-160-D11==protein tyrosine/serine/threonine phosphatase
002-145-H09==protein-methionine-S-oxide reductase
006-310-C10==pseudouridylate synthase
J013069D01==pyridoxal kinase
J033070K02==pyridoxamine-phosphate oxidase
006-305-H12==pyroglutamyl-peptidase I
J023036A18==pyrophosphatase
J023044E03==pyrroline 5-carboxylate reductase
002-149-C12==pyruvate kinase
J033096C17==queuine tRNA-ribosyltransferase
J023100009==RAB GDP-dissociation inhibitor
J023076015==RAB small monomeric GTPase
002-145-H05==“racemase and epimerase, acting on amino acids and derivatives”
002-163-F07==RAS GTPase activator
001-103-H08==RAS small monomeric GTPase
J023053H09==recombinase
J013169B12==ribonucleoside-diphosphate reductase
J013026J06==ribose-5-phosphate isomerase
J023042N11==ribulose-bisphosphate carboxylase
006-205-A08==ribulose-phosphate 3-epimerase
002-174-A05==“rRNA (adenine-N6,N6-)-dimethyltransferase”
002-143-D09==S-adenosylmethionine-dependent methyltransferase
002-183-A05==serine carboxypeptidase
002-146-H03==serine esterase
J023104M20==serine-tRNA ligase
J023048E15==serine-type endopeptidase
001-103-F08==serine-type peptidase
002-146-B12==shikimate kinase
J023147K09==sialyltransferase
J023012K16==small monomeric GTPase
002-153-C06==stearoyl-CoA desaturase
002-145-D10==strictosidine synthase
002-147-B12==subtilase
002-144-D08==succinate dehydrogenase
J013043008==sulfate adenylyltransferase (ATP)
J023042G18==sulfotransferase
J023060D13==superoxide dismutase
J023133M06==thiamin-phosphate pyrophosphorylase
J013065E05==thiosulfate sulfurtransferase
J013057I21==thymidine kinase
002-146-A10==transaminase
J023057D05==transferase
J023035J06==“transferase, transferring glycosyl groups”
J023034P03==“transferase, transferring hexosyl groups”
J033065A07==“transferase, transferring phosphorus-containing groups”
002-164-E12==transketolase
J023048G03==transmembrane receptor protein tyrosine kinase
001-118-H08==triacylglycerol lipase
J023115C24==triose-phosphate isomerase
J033124K13==tRNA
(5-methylaminomethyl-2-thiouridylate)-methyltransferase
002-148-C06==tRNA ligase
006-206-E08==tRNA-intron endonuclease
002-154-F02==trypsin
J023125E11==tryptophan synthase
001-046-E04==ubiquinol-cytochrome c reductase
J023049Il2==ubiquitin activating enzyme
J023055G02==ubiquitin conjugating enzyme
J013066H16==ubiquitin C-terminal hydrolase
J023039K21==ubiquitin-protein ligase
J013071N01==UDP-3-O-[3-hydroxymyristoyl]
N-acetylglucosamine deacetylase
J023089L18==urate oxidase
J033082E21==uridine kinase
002-124-B12==uroporphyringonen-III synthase
J023047E18==uroporphyrinogen decarboxylase
J023078I01==UTP-hexose-1-phosphate uridylyltransferase
J023088P07==vacuolar aminopeptidase I
J023028013==voltage-gated chloride channel
002-127-C10==xylose isomerase
   “List of Clones Derived From Flower Organs”

This list (Table 17; submitted in electronic format) shows the relationship between the names of respective clones derived from flower organs and the libraries from which the clones are derived.

What is claimed is:

1. An isolated plant-derived nucleic acid, wherein said nucleic acid is selected from the group consisting of:
(a) a nucleic acid encoding a protein comprising an amino acid sequence set forth in any one of SEQ ID NOs: 28470 through 56791;
(b) a nucleic acid containing the coding region of a nucleotide sequence set forth in any one of SEQ ID NOs: 1 through 28469;
(c) a nucleic acid encoding a protein comprising an amino acid sequence set forth in any one of SEQ ID NOs: 28470 through 56791 wherein one or more amino acids are substituted, deleted, inserted and/or added; and
(d) a nucleic acid hybridizing to a nucleic acid comprising a nucleotide sequence set forth in any one of SEQ ID NOs: 1 through 28469 under stringent conditions.

2. The nucleic acid according to claim 1, wherein said nucleic acid is derived from rice.

3. An isolated DNA molecule selected from the group consisting of:
(a) a DNA molecule encoding an antisense RNA complementary to a transcript of the DNA molecule of claim 1;
(b) a DNA molecule encoding RNA having ribozyme activity to specifically cleave a transcript of the DNA of claim 1;
(c) a DNA molecule encoding RNA inhibiting the expression of the DNA of claim 1 via an RNAi effect at the time of expression of said DNA in plant cells; and
(d) a DNA molecule encoding RNA inhibiting the expression of the DNA of claim 1 by the co-suppression effect at the time of expression of said DNA in plant cells.

4. An isolated DNA molecule selected from the group consisting of:
(a) a DNA molecule encoding an antisense RNA complementary to a transcript of the DNA molecule of claim 2;
(b) a DNA molecule encoding RNA having ribozyme activity to specifically cleave a transcript of the DNA of claim 2;
(c) a DNA molecule encoding RNA inhibiting the expression of the DNA of claim 2 via an RNAi effect at the time of expression of said DNA in plant cells; and
(d) a DNA molecule encoding RNA inhibiting the expression of the DNA of claim 2 by the co-suppression effect at the time of expression of said DNA in plant cells.

5. A vector containing the nucleic acid of claim 1.

6. A vector containing the nucleic acid of claim 2.

7. A vector containing the nucleic acid of claim 3.

8. A vector containing the nucleic acid of claim 4.

9. A transformed plant cell maintaining the nucleic acid of claim 1.

10. A transformed plant cell maintaining the nucleic acid of claim 3.

11. A transformed plant cell maintaining the nucleic acid of claim 4.

12. A transformed plant cell maintaining the vector of claim 5.

13. A transformed plant body containing the transformed plant cell of claim 9.

14. A progeny or clone of the transformed plant body of claim 13.

15. A propagation material of the transformed plant body of claim 13.

16. A propagation material of the transformed plant body of claim 14.

17. A method of producing a transformed plant body, wherein said method comprises the step of transducing the nucleic acid of claim 1 into plant cells to regenerate a plant body from said plant cells.

18. A method of producing a transformed plant body, wherein said method comprises the step of transducing the nucleic acid of claim 3 into plant cells to regenerate a plant body from said plant cells.

19. A method of producing a transformed plant body, wherein said method comprises the step of transducing the nucleic acid of claim 4 into plant cells to regenerate a plant body from said plant cells.

20. A method of producing a transformed plant body, wherein said method comprises the step of transducing the vector of claim 5 into plant cells to regenerate a plant body from said plant cells.

21. A protein encoded by the nucleic acid of claim 1.

22. A method of producing a protein encoded by the nucleic acid of claim 1 comprising the following steps:
(1) transducing the nucleic acid of claim 1 or a vector containing said nucleic acid into cells capable of expressing said nucleic acid so as to obtain a transformant;
(2) culturing said transformant; and
(3) recovering the protein from the culture of the step (2).

23. An antibody binding to the protein of claim 21.

25. A rice gene database comprising sequence information selected from the group consisting of:
(a) one or more amino acid sequences selected from SEQ ID NOs: 28470 through 56791;
(b) one or more nucleotide sequences selected from SEQ ID NOs: 1 through 28469; and
(c) both (a) and (b).

26. A method of determining the transcriptional regulatory region comprising the steps of:
(1) mapping the nucleotide sequence of any one of SEQ ID NOs: 1 through 28,469 to the rice genome nucleotide sequence, and
(2) determining the transcriptional regulatory region of the gene mapped in the step (1) which contains the transcriptional regulatory region found on the 5′-side of the 5′ most end of the mapped region.
